(19) United States
(12) Patent Application Publication
(10) Pub. No.: US 2014/0242581 A1
(43) Pub. Date: Aug. 28, 2014

(54) COMPOSITIONS AND METHODS FOR GENETIC ANALYSIS OF EMBRYOS

(71) Applicant: Reproductive Genetics and Technology Solutions, LLC, Los Angeles, CA (US)

(72) Inventor: Mark T. Johnson, Santa Monica, CA (US)

(73) Assignee: Reproductive Genetics and Technology Solutions, LLC, Los Angeles, CA (US)

(21) Appl. No.: 14/162,466

(22) Filed: Jan. 23, 2014

Related U.S. Application Data
(60) Provisional application No. 61/755,760, filed on Jan. 23, 2013.
(60) Provisional application No. 61/785,752, filed on Mar. 14, 2013.

(51) Int. Cl. C12Q 1/68

(52) U.S. Cl. CPC C12Q 1/6869
USPC 435/6.11

(57) ABSTRACT

The present disclosure provides for compositions and methods for genetic analysis of embryos. Generally, the compositions and methods provide for the acquisition of an sample containing RNA from an embryo, genetic analysis involving various techniques such as sequencing-, hybridization- or amplification-based methods, and the detection of genetic alterations that may affect the health and quality of the embryo. In some cases, compositions and methods of this disclosure may provide information useful in the selection and monitoring of embryos for implantation into a female.

### CROSS-REFERENCE

This application claims the benefit of U.S. Provisional Application No. 61/755,760, filed Jan. 23, 2013 and U.S. Provisional Application No. 61/785,752, filed Mar. 14, 2013, which applications are incorporated herein by reference in their entireties.

### BACKGROUND OF THE DISCLOSURE

Human embryos generated through assisted reproductive technologies (ART) are prone to various genetic alterations, including copy number variations (CNV) that involve entire or large segments of chromosomes. Recent studies of human embryos generated in vitro through assisted reproductive technologies (ART) have shown that more than half of embryos generated from couples contain at least some cells with these large CNVs. These abnormalities cannot be attributed solely to infertility as there is also a high rate of large CNVs in couples who are young and presumably fertile. Most of these large CNVs arise as a result of errors in the meiotic divisions and early embryonic mitotic divisions (FIG. 1).

Large CNVs have a tremendous negative impact on human fecundity and well-being. Most aneuploidies lead to early prenatal demise as evidenced by the findings that more than 35% of spontaneous abortions karyotyped are found to be aneuploid and, almost 70% are found to have large genomic imbalances by using higher resolution microrrays. Based on these data and the finding that many genomic abnormalities reported in early embryos are never seen in later prenatal or postnatal periods, it is clear that most of these large CNVs are eliminated in early pregnancy. These abnormalities also represent a major cause of failed ART cycles, which are those that do not lead to a pregnancy or livebirth. However, not all large CNVs lead to early pregnancy loss. A small subset of aneuploidies, including a few trisomies and sex chromosome aneuploidies, are compatible with development to the later prenatal stages or even beyond birth. Approximately 8% of stillbirths and 0.3% of liveborn children are aneuploid. Postnatally, aneuploidy is the most common recognized genetic cause of mental retardation.

Current approaches to screen for CNVs in preimplantation human embryos focus on determining the copy number of genomic DNA isolated from biopsied cells. These methods have the limitations that there is a considerable test failure rate and the biologic information obtained from analysis of DNA is fairly limited. Even when human embryos have been successfully screened for CNVs using DNA-based approaches, a substantial proportion, often the majority, of embryos still do not produce healthy, liveborn offspring. An RNA-based approach for screening for CNVs has the advantages relative to DNA-based screening that may include but are not limited to: (1) analyses of transcripts in samples containing few cells are likely to be more successful since, for many loci, there are many more copies of the transcript than the copies of the genomic locus, (2) by focusing on the transcribed region of the genome, the complexity of the sample is reduced, a particular advantage for sequencing based methods since the read coverage can be increased, (3) RNA is much less stable than DNA, which reduces the likelihood of contamination from exogenous nucleic acids, and (4) transcriptome analysis provides much more information content pertaining to the health and physiology of cells than DNA does. There is need in the art for improved screening methods for CNVs.

### SUMMARY OF THE DISCLOSURE

This disclosure provides compositions and methods for detecting genetic alterations in embryos. Generally, this disclosure provides for compositions and methods for determining a presence or absence of a genetic alteration in an embryo, wherein the method comprises analysis of RNA. In some aspects, the embryo analyzed will be a mammalian embryo in the preimplantation period of development. In some aspects, these genetic alterations will be copy number variants within the genome.

In some embodiments, this disclosure provides compositions and methods for determining a presence or absence of a genetic alteration in an embryo, wherein the method comprises reverse transcribing RNA from the embryo to form cDNA and analyzing the cDNA.

In some embodiments, this disclosure provides compositions and methods for determining a presence or absence of a genetic alteration in an embryo, wherein the method comprises analysis of amplification products of cDNA derived from the embryo.

In some aspects, this disclosure provides compositions and methods comprising identification and quantitation of RNAs expressed from the embryo.

In some aspects, these methods of analyzing embryos may detect other genetic abnormalities such as mutations and causal variants. In other aspects these methods may detect epigenetic alterations.

In some embodiments, all nucleic acids derived from the embryo are analyzed. In other embodiments a subset of nucleic acids derived from the embryo are analyzed.

In some embodiments, the nucleic acids derived from the embryo are analyzed to determine the levels of transcripts in a pre-defined window to generate a regional expression count.

In some embodiments, regional expression counts from one or more pre-defined regions from the embryo are compared to similar regions in a reference to generate relative regional expression values.

In some embodiments, regional expression counts from one or more pre-defined regions from the embryo are compared to similar regions in a reference and evaluated using statistical analyses to generate relative regional expression values.

In some embodiments, the method further comprising analysis of the relative regional expression values of pre-defined regions in the embryo are further analyzed statistically to determine a presence or absence of a genome copy number variation.

In some embodiments the pre-defined regions used for generating regional expression values may be an exon, gene, locus, transcription unit, region of defined of length, and allele.

In some embodiments, the reference comprises one preimplantation embryo. In other embodiments the reference comprises more than 1, 10, 100 or 1000 preimplantation embryos with known or unknown genotypes.

In some embodiments, the analyses are performed using one or more computer executable algorithms.

In some embodiments, the nucleic acids derived from the embryo are analyzed by sequencing. Sequencing includes the steps of generating sequence, aligning sequence to a reference sequence, and enumerating the number of reads in pre-defined regions of the reference sequence to generate regional expression counts that can be further analyzed using the algorithms described herein. In some aspects, sequencing further comprises analysis of the whole transcriptome or partial transcriptome.

In some embodiments, the nucleic acids derived from the embryo are analyzed by hybridization to one or more probes. In some cases these probes are included in a microarray. The number of target sequences that anneal to the probes are quantitated over a pre-defined region of a reference sequence to generate regional expression counts that can be further analyzed as described herein.

In some embodiments, the nucleic acids derived from the embryo are analyzed by amplification. In some cases, quantitative PCR or digital PCR are used for quantitation. The estimated amount of template sequences that are contained within predefined regions are used to generate regional expression counts that can be further analyzed using methods described herein.

In some embodiments the RNA is obtained from the embryo invasively in which cells or subcellular compartments are removed from the embryo. In other embodiments, the sample is obtained noninvasively by collecting cells or cell-free RNA from liquid surrounding the embryo.

In some embodiments, the sample derived from the pre-implantation embryo is collected less than 1 hour, 6 hours, 12 hours, 1 day, 2 days, 3 days, 4 days, 5, days, 6 days, 7 days, 8 days, 9 days, 10 days, 2 weeks or 3 weeks after the initiation of RNA expression in the pre-implantation embryo or after fertilization of the pre-implantation embryo.

In some embodiments, the embryo is a mammalian embryo in the preimplantation period that has been generated by fertilization in vivo or in vitro. The preimplantation period is considered to encompass the period that begins with fertilization and extends to the latest timepoint at which an embryo can be maintained in vitro and still have a possibility of producing a healthy liveborn following transfer to the female. In some embodiments, the embryo is a human embryo. In some instances, the embryo is at the blastocyst stage.

In some embodiments, the embryo is generated in vitro from one or more oocytes derived from a female following stimulation of the female with exogenous hormones.

In some embodiments, the genetic alteration detected is copy number variation involving all of part of a chromosomal abnormality. In some instances, these abnormalities correlate with the developmental potential.

In some embodiments, the analysis also assess epigenetic status, determines sex, exposure to stress or toxins, metabolism or mitochondrial load.

This disclosure provides compositions and methods for genetic analysis of embryos. Generally, this disclosure provides for compositions and methods for determining a presence or absence of a genomic copy number variation in a preimplantation embryo, wherein the method comprises analysis of RNA.

In some aspects, this disclosure provides compositions and methods comprising high-throughput sequencing of RNA from the preimplantation embryo. In some aspects, sequencing further comprises analysis of the whole transcriptome or partial transcriptome.

In some aspects, determining a presence or absence of a genomic copy number variation in a preimplantation embryo, comprises reverse transcribing RNA derived from a preimplantation embryo to form cDNA and analyzing the cDNA to determine a presence or absence of the genomic copy number variation in the preimplantation embryo.

In some embodiments, sequencing further comprises enumerating sequence reads, aligning the sequence reads to a reference genome, and comparing a number of the sequence reads corresponding to one or more loci on a first chromosome to a number of the sequence reads corresponding to one or more loci on a second chromosome, wherein the first chromosome is suspected of exhibiting a copy number variation, and the second chromosome is euploid. Sequencing may be performed on any RNA or cDNA.

In some embodiments, analysis of sequence reads further comprises normalizing a number of the sequence reads corresponding to one or more loci on a first chromosome suspected of exhibiting a copy number variation to generate a normalized chromosome count, and comparing the normalized chromosome count to a normalized chromosome count for a reference sample from one or more embryos without a genomic imbalance. In some aspects, a number of the sequence reads corresponding to one or more loci on a first chromosome suspected of exhibiting a copy number variation is normalized to a number of the sequences reads corresponding to one or more loci on a second chromosome suspected of being euploid. In some aspects, wherein the number of the sequences reads corresponding to one or more loci on a first chromosome suspected of exhibiting a copy number variation is normalized to a number of the sequence reads corresponding to loci on a plurality of chromosomes. In some cases analysis comprises use of statistical analysis which may be conducted using an algorithm executed by an computer.

In some embodiments of this disclosure, RNA or cDNA is analyzed through hybridizing the RNA to a microarray, in vitro transcription of the cRNA, amplifying all RNA or cDNA, amplifying selected RNA or DNA, amplifying random RNA or cDNA and amplifying non-random RNA or cDNA.

In some embodiments, wherein a plurality of preimplantation embryos is analyzed, RNA is amplified from the plurality of preimplanation embryos and individual RNAs or cDNAs are indexed, such as with the attachment of a barcode sequence.

In some aspects, analyzing RNA from preimplantation embryos comprises annealing a plurality of probe-pairs to a plurality of individual RNA molecules, wherein each probe-pair comprises a capture probe capable of annealing to an individual RNA or cDNA and a reporter probe capable of annealing to the individual RNA.

In some aspects, analyzing RNA from preimplantation embryos comprises comparing an amount of RNA, median expression value of RNA or cDNA, or normalized expression value for RNA or cDNA, derived from one or more loci to an amount of RNA or cDNA, median expression value, or median value of RNA or cDNA, derived from the one or more loci from one or more embryos known to be euploid or disomic for the one or more loci.

In some embodiments, analyzing RNA from preimplantation embryos comprises determining a first ratio of an amount of RNA or cDNA derived from a first set of one or more loci to an amount of RNA or cDNA derived from a second set of one or more loci, and comparing the first ratio to a second ratio derived from one or more embryos known to be euploid, wherein the second ratio is a ratio of an amount of RNA or cDNA derived from the first set of one or more loci to an amount of RNA or cDNA derived the second set of one or more loci.

In some embodiments, analyzing RNA from preimplantation embryos comprises determining a first ratio of an amount of RNA or cDNA derived from a first set of one or more loci to an amount of RNA or cDNA derived from a second set of one or more loci, and comparing the first ratio to a second ratio derived from a plurality of embryos, wherein the second ratio is a ratio of an amount of RNA or cDNA derived from the first set of one or more loci to an amount of RNA or cDNA derived from the second set of the one or more loci.

In some aspects, statistical analysis is performed to determine the presence or absence of a copy number variation.

In some aspects analyzing RNA from preimplantation embryos comprises comparing an amount of RNA or cDNA derived from one parental allele corresponding to one or more loci on a chromosome to an amount of RNA or cDNA derived from the other parental allele corresponding to the one or more loci on the chromosome to determine an allele ratio, and comparing the ratio to a reference ratio to determine a presence or absence of a copy number variation of one of the alleles.

In some aspects analyzing RNA from preimplantation embryos comprises comparing an amount of RNA or cDNA derived from one parental allele corresponding to one or more loci on a chromosome to an amount of RNA or cDNA derived from the same parental allele from one or more samples known to have a single copy of the allele.

In some aspects analyzing RNA from preimplantation embryos comprises comparing an amount of RNA or cDNA derived from one parental allele corresponding to one or more loci on a chromosome to a median amount of the RNA or cDNA derived from the same parental allele from one or more samples known to have a single copy of the allele.

In some aspects analyzing RNA from preimplantation embryos comprises determining a ratio of parental alleles of one or more loci, and comparing the ratio to a ratio of parental alleles of the one or more loci from one or more embryos known to have a single copy of each allele. In some aspects, analyzing RNA or cDNA from preimplantation embryos comprises determining a ratio of parental alleles of one or more loci, and comparing the ratio to a ratio of paternal alleles of the one or more loci from a plurality of embryos. In some aspects, copy number variation comprises loss of heterozygosity.

In some embodiments, this disclosure provides for RNA which comprises transcribed RNA, messenger RNA, noncoding RNA, a plurality of RNA transcripts, or a plurality of random RNA transcripts.

In some embodiments, analyzing RNA from preimplantation embryos comprises preparing a report based on the analysis and sending the report to a subject. In some aspects, one or more preimplantation embryos are selected and placed in a uterus of the female based on the analysis.

In some embodiments, selection and placement of preimplantation embryos is at the blastocyst stage. Selection of preimplantation embryos may or may not further comprise analyzing the morphology of the preimplantation embryo. Selection of preimplantation embryos may or may not further comprise analyzing genomic DNA of the preimplantation embryo. Preimplantation embryos may be frozen, before or after selection for implantation.

In some aspects, preimplantation embryos are analyzed, comprising performing secretome and metabolic profiling of culture media in which the preimplantation embryo is cultured.

In some embodiments, the preimplantation embryo is generated from an oocyte from the female, from an oocyte derived from ovarian tissue cultured in vivo, from an oocyte derived from a germ cell in vitro, from an oocyte derived from a stem cell, or from an oocyte from a second female, wherein the female receiving the preimplantation embryo and the second female are not the same female.

In some embodiments, the preimplantation embryo is generated by in vitro fertilization, or intracytoplasmic sperm injection.

In some aspects, expression level of one or more genes is determined from the RNA or cDNA of preimplantation embryos. In some aspects, the expression level of the one or more genes correlates with embryonic health or developmental potential of the preimplantation embryo. In some aspects, the epigenetic status of the genome of the preimplantation embryo is determined.

In some embodiments, analyzing RNA from a preimplantation embryo comprises determine a sex, (i.e. male or female) of the preimplantation embryo.

In some embodiments, analyzing RNA from a preimplantation embryo comprises determining expression patterns of loci associated with one or more responses to environmental stress, further comprising presence of a toxin, high or low temperature, high oxygen, oxidative stress, high or low osmolarity, or inadequate nutrition.

In some embodiments, analyzing RNA from a preimplantation embryo comprises determining expression patterns of loci associated with metabolism. In some cases analyzing RNA from a preimplantation embryo comprises determining expression patterns of mitochondrial loci, which may further comprise assessing mitochondrial load or assessing metabolic activities.

In some embodiments, analyzing RNA from a preimplantation embryo comprises analyzing expression levels of genes whose expression is modulated by a copy number variation. Analysis may further comprise determining a presence or absence of one or more mutations in one or more genes or linkage analysis.

In some aspects, one embryo is analyzed. In some aspects a plurality of embryos is analyzed. An embryo may further comprise a mammalian embryo, a human embryo, a domestic animal embryo, or an endangered animal embryo.

In some aspects, the embryo is a human embryo, and the copy number variation is an aneuploidy involving chromosome 13, 18, 21, X, or Y. In some cases, aneuploidy is a trisomy, such as trisomy 13, trisomy 18, or trisomy 21. In some cases, copy number variation is a monosomy.

In some cases, RNA is derived from cells, or subcellular compartments, such as a nucleus, cytoplasm, or from cell free sources, such as bodily fluids or embryo culture media.

In some aspects, high-throughput sequencing of RNA or cDNA derived from embryos comprises bridge amplification and incorporation of four fluorescently-labeled, reversible terminator-bound dNTPs; measurement of release of inorganic phosphate; passing the cDNA through a nanopore; or measuring hydrogen ion release during polymerization of cDNA.

In some aspects, analysis of RNA or cDNA derived from embryos comprises hybridizing the cDNA to a microarray, amplifying the cDNA, performing PCR, real-time PCR, isothermal amplification, linear amplification, or isothermal linear amplification.

In some aspects, preimplantation embryos may be selected based on analysis, and transferred to the uterus of a female. In some cases, the embryo is at the blastocyst stage. In some cases, the morphology, or genomic DNA or epigenetic status of the preimplantation is also analyzed.

In some cases, analysis is performed on RNA derived from a maternal sample, such as blood.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of a device of this disclosure are set forth with particularity in the appended claims. A better understanding of the features and advantages of this disclosure will be obtained by reference to the following detailed description that sets forth illustrative embodiments, in which the principles of a device of this disclosure are utilized, and the accompanying drawings of which:

FIG. 1 is a schematic representation of the origins of genomic imbalances, such as aneuploidies and segmental aneusomy, which are alterations in the copy number of segments of chromosomes, during various stages of development of an embryo.

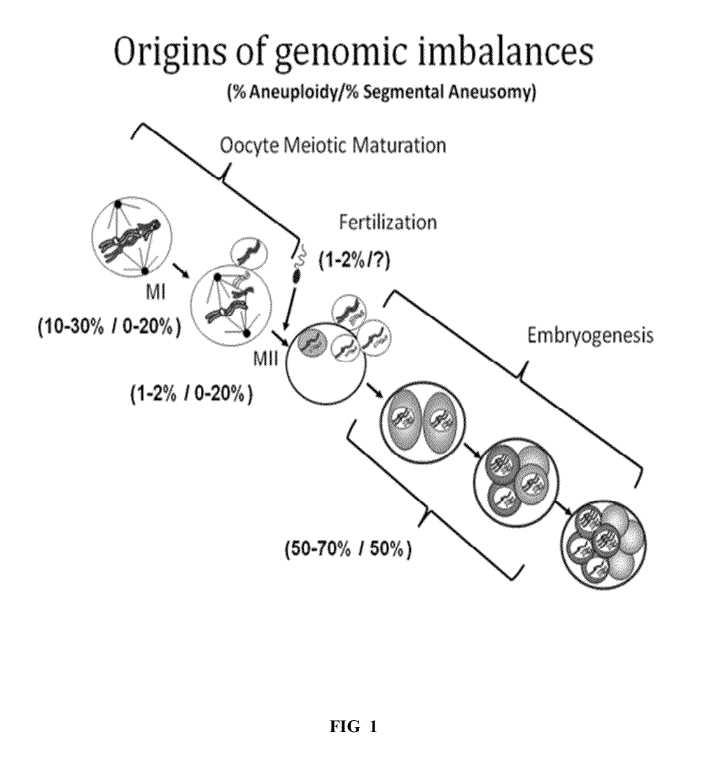

FIG. 2 is a schematic flow diagram of exemplary steps of a general workflow as described by disclosure.

FIG. 3 is a series of photographs of embryo biopsy techniques at various development stages of human embryos.

FIG. 4 is a schematic diagram of types of nucleic acids that can be generated from RNA samples and the types of nucleic acids that can be analyzed. Amplification is abbreviated as ‘Amp.’

FIG. 5 is a schematic of several different protocols that have been used to prepare libraries for massively parallel sequencing of amplified sequences derived from RNA from single cells.

FIG. 6 is a schematic of data processing and analysis steps for transcriptome data generated by high throughput sequencing, hybridization or Amplification-based methods.

FIG. 7 is an exemplary diagram of storage and dissemination of transcriptome analysis results via computer.

FIG. 8 is a schematic representation of workflow for the preparation of libraries using the Smart-Seq approach.

FIG. 9 is a schematic illustration of the tn5-transposase Nextera method for simultaneously fragmenting DNA and ligating adaptors to the ends of the fragments.

FIG. 10 is a schematic representation of workflow for identifying CNVs from sequencing data using the ExomeCNV software package.

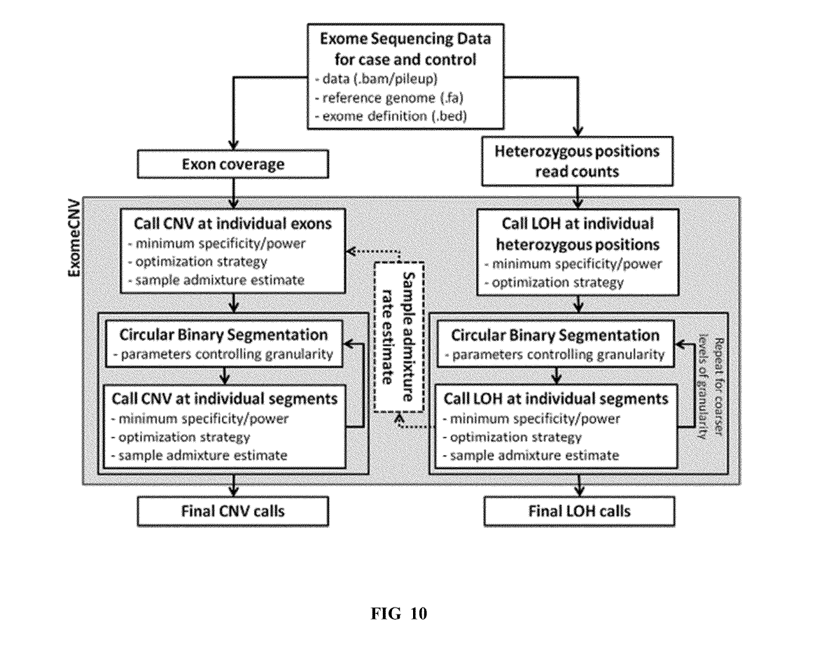

FIG. 11 is a diagram showing how one form of meiotic segregation involving double Robertsonian translocations with monobrachial homology (white chromosomes) can lead to aneuploidies of the monobrachial chromosome.

FIG. 12 is a representation of the workflow for generating, assessing the development of, genotyping, and isolating RNA samples from aneuploid mouse embryos.

FIG. 13 is a Manhattan plot representing the fold changes in loci expression from mouse embryos with trisomy 10 as compared to normal disomic samples. The data are binned by chromosome number along the abscissa.

### DETAILED DESCRIPTION OF THE DISCLOSURE

### I. General Terminology

The compositions and methods of this disclosure as described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3^{rd} Ed., W.H. Freeman Pub., New York (2000); and Berg et al., Biochemistry, 5^{th} Ed., W.H. Freeman Pub., New York (2002) and Rodriguez-Ezpeleta Bioinformatics for High Throughput Sequencing, Springer, New York (2012), all of which are herein incorporated by reference in their entirety for all purposes. Before the present compositions, research tools and methods are described, it is to be understood that this disclosure is not limited to the specific methods, compositions, targets and uses described, as such may, of course, vary. It is also to be understood that the terminology used herein is for the purpose of describing particular aspects only and is not intended to limit the scope of the present disclosure, which will be limited only by appended claims.

The terminology used herein is for the purpose of describing particular embodiments only and is not intended to be limiting of a device of this disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Several aspects of a device of this disclosure are described above with reference to example applications for illustration. It should be understood that numerous specific details, relationships, and methods are set forth to provide a full understanding of a device. One having ordinary skill in the relevant art, however, will readily recognize that a device can be practiced without one or more of the specific details or with other methods. This disclosure is not limited by the illustrated ordering of acts or events, as some acts may occur in different orders and/or concurrently with other acts or events. Furthermore, not all illustrated acts or events are required to implement a methodology in accordance with this disclosure.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### II. Overview

The present disclosure provides for compositions and methods for identifying genetic alterations using RNA obtained from embryos. Genetic alterations encompass any changes in genomic sequence relative to another sequence, typically a reference sequence. Genetic alterations include mutations, which are considered to cause disease, and polymorphisms, which are alterations present in greater than 1% of the population. Genetic alterations include, but are not limited to, point mutations, transversions, transitions, nonsense mutations, frame shift mutations, repeat mutations, indels, deletions, translocations, inversions and duplications, SNPs, CNVs and simple sequence repeats. These alterations may cause genetic disease, contribute to susceptibility of disease or contribute to a variety of traits. It is estimated that 85% of disease causing mutations are in the coding region of the genome. Any alterations that are located within the coding region of loci that are transcribed in the embryo may be detected directly. Other mutations may be detected indirectly either through linkage analysis or direct or indirect effects on expression of one or more transcripts.

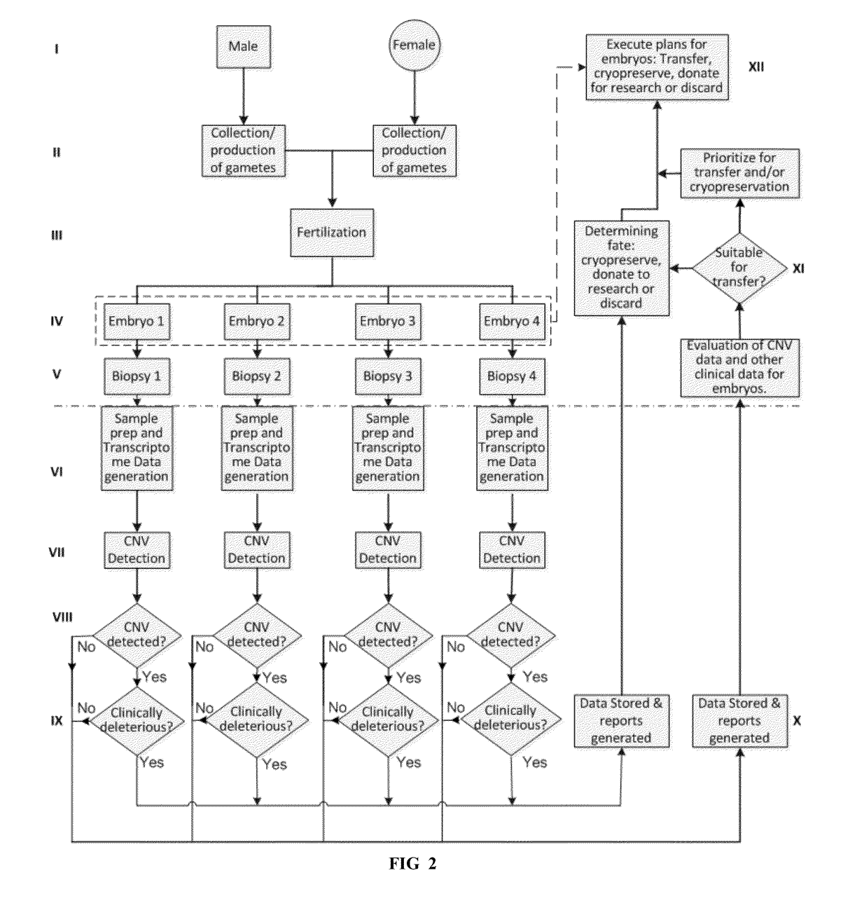

The present disclosure provides for compositions and methods for identifying genetic CNVs through analysis of RNA obtained from embryos. The principle behind CNV detection is based on the observation, described in detail in Example 1, that there is a high correlation between the level of RNA produced from a locus and the number of copies of this locus within the genome in embryos within days after the initiation of expression of the embryonic genome. Based on this finding, CNVs can be detected in early embryos by identifying regional disturbances in the expression of the loci. For example, a trisomy, a condition in which there is an extra copy of a chromosome, can be detected due to increased expression of many of the loci on the trisomic chromosome. There are many potential applications for CNV screening of early embryos. One application of clinical relevance would be to screen human embryos that have been generated in vitro by assisted reproductive technologies for CNVs before they are transferred to the uterus to establish a pregnancy. A schematic workflow for human embryo CNV screening is shown in FIG. 2.

CNV screening involves a multitude of steps to go from an embryo to a diagnostic result. The first step is the generation or retrieval of an embryo for sampling. A sample containing RNA must then be obtained. A number of potential processing steps may then be performed on the sample to generate the appropriate form and sufficient amounts of material for analysis. A number of analytic methods may be used to determine the levels of multiple RNAs in the sample. The methods are divided into sequencing-, hybridization- and amplification-based approaches. Following generation of the raw data from these analytic methods, the data are analyzed to identify CNVs. The identified genetic abnormalities may impact the health of the embryo, its subsequent development or health at later stages of development. In some cases, compositions and methods of this disclosure may provide information useful in the selection and monitoring of embryos for implantation into a female.

### III. Embryo Generation

The source of samples for the compositions and methods of this disclosure is an embryo from any species at any stage after there is expression of RNA encoded by the genome of the embryo. An embryo may be from a vertebrate or an invertebrate, preferably a mammal. A mammalian embryo may from a human, a non-human primate, livestock, cow, horse, pig, sheep, goat, cat, buffalo, guinea pig, hamster, rabbit, mice, domesticated species and endangered species. In most cases, these diagnostic approaches will be applied within days or weeks following the initiation of expression of the embryonic genome. For mammalian species, the early stages of the embryo that precede implantation into the uterine wall are referred to as the preimplantation period. For human embryos, the natural preimplantation period extends from the time the oocyte is fertilized until the beginning of implantation, a period of about 6 days. The preimplantation period also encompasses the following developmental stages: zygote, cleavage-stage embryo, morula, blastocyst, 1-2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, 50-, 51-, 52-, 53-, 54-, 55-, 56-, 57-, 58-, 59-, 60-, 61-, 62-, 63-, 64-, 65-, 66-, 67-, 68-, 69-, 70-, 71-, 72-, 73-, 74-, 75-, 76-, 77-, 78-, 70-, 80-, 81-, 82-, 83-, 84-, 85-, 86-, 87-, 88-, 89-, 90-, 91-, 92-, 93-, 94-, 95-, 96-, 978-, 98-, 99-, 100-, 101-, 102-, 103-, 104-, 105-, 106-, 107-, 108-, 109-, 110-, 111-, 112-, 113-, 114-, 115-, 116-, 117-, 118-, 119-, 120-, 121-, 122-, 123-, 124-, 125-, 126-, 127-, 128-, 129-, 130-, 131-, 132-, 133-, 134-, 135-, 136-, 137-, 138-, 139-, 140-, 141-, 142-, 143-, 144-, 145-, 146-, 147-, 148-, 149-, 150-, 151-, 152-, 153-, 154-, 156-, 157-, 158-, 159-, 160-, 161-, 162-, 163-, 164-, 165-, 166-, 167-, 168-, 169-, 170-, 171-, 172-, 173-, 174-, 175-, 176-, 177-, 178-, 179-, 180-, 181-, 182-, 183-, 184-, 185-, 186-, 187-, 188-, 189-, 190-, 191-, 192-, 193-, 194-, 195-, 196-, 197-, 198-, 199- and 200-cell embryo.

The focus of this application is on developmental stages at which the embryo can be generated and maintained in vitro and still allow for a healthy pregnancy to be established following the transfer of the embryo into the female. Should techniques allow for the embryo to be maintained in culture for a longer period than the natural preimplantation period, then this period will also be considered to be the preimplantation period. This period could conceivably be extended for days or even weeks. When embryos are cryopreserved, the period of time during which the embryo is cryopreserved is not considered to be part of the preimplantation period since the embryos are in a state of suspended animation.

The compositions and methods of this disclosure provide for embryos that may be generated by any means capable of producing a healthy, normal liveborn offspring. Various techniques for generation of embryos are well known in the art, examples of which are incorporated by reference herein (see Clinical Gynecologic Endocrinology and Infertility Fritz, M. and Speroff, L. Eds; Philadelphia: Lippincott Williams & Wilkins (2010) and Textbook of assisted reproductive techniques: laboratory and clinical perspectives Gardner, D. K, et al, London: CRC Press (2012), incorporated herein by reference).

### III.A. Oocyte Generation

Before the generation of embryos, female gametes, or oocytes, must be retrieved from the female or produced by a method that generates an oocyte capable of being fertilized and supporting the production of a healthy liveborn. The term “oocyte” refers to the gamete from the follicle of a female animal, whether vertebrate or invertebrate. The animal is preferably a mammal, including a human, non-human primate, cow, horse, pig, sheep, goat, cat, buffalo, guinea pig, hamster, rabbit, mice, domesticated species and endangered species. Suitable oocytes for use in the disclosure may include but are not limited to immature oocytes, and mature oocytes from ovaries stimulated by administering a fertility agent(s) or fertility enhancing agent(s) (e.g. inhibin, inhibin and activin, clomiphene citrate, human menopausal gonadotropins including FSH, or a mixture of FSH and LH, and/or human chorionic gonadotropins) to the oocyte donor or the obtained specimen. In some embodiments of the disclosure, the oocytes are aged (e.g. from humans 40 years+, or from animals past their reproductive prime). Methods for isolating oocytes are known in the art, examples of which are described herein.

In some cases, oocytes may be obtained through a controlled ovarian stimulation protocol to promote ovarian follicle growth and maturation. For example, in humans, hormonal treatment cycles generally begin on the third day of menstruation, constituting about ten days of daily subcutaneous injections of gonadotropins. These injections may consist of protein hormones, termed gonadotropins, utilized under close monitoring. This monitoring frequently involves evaluating the estradiol hormone levels and ovarian follicular growth. The prevention of spontaneous ovulation involves utilization of other hormones such as GnRH antagonists or GnRH agonists that block the natural surge of luteinizing hormone. A protocol individualized for patients based on response to hormones and history may be employed. Alternatively, oocytes may be retrieved using minimal stimulation or during natural cycles (i.e., no exogenous hormonal stimulation). When follicles are of the proper stage of development for retrieval, typically just prior to ovulation, the oocytes may be retrieved using known methods such as transvaginal, ultrasound-guided follicular aspiration. In other cases, the follicles are aspirated by perurethral/transvesical ultrasonographic puncture. In other cases, the oocytes are retrieved laparoscopically. Once the follicular fluid is removed from the follicle, the eggs are located within the fluid using microscopy, inspected, and suitable specimens are placed into culture medium in an incubator. Oocytes may also be cryopreserved if the fertilization is to be performed at a later date.

Another example method of generating oocytes as provided by the composition and methods of this disclosure is to obtain immature follicles or oocytes and mature them in vitro under conditions such as those used in the art to promote oocyte maturation (see patents U.S. Pat. No. 5,882,928 and U.S. Pat. No. 6,281,013, incorporated by reference herein).

Another example method of obtaining oocytes may comprise isolating oocytes that have developed from ovarian stem cells isolated from one or more ovaries (see White, et al. (2012) Nature Medicine 18: 413-422, incorporated by reference herein).

Another method of obtaining oocytes may be through the acquisition of ovarian tissue followed by culture in vitro or transplantation, autologous or heterologous. In some cases, the ovarian tissue may be cryopreserved prior to culture or transplantation.

### III.B. Sperm Generation

Additionally, male gametes (i.e., sperm) are obtained for embryo generation. Methods for isolating male gametes are known in the art. Male gametes may be retrieved by ejaculation as a result of intercourse, masturbation, electrical or vibratory stimulation to the prostate or penis, puncture of the spermatic ducts or testicle biopsy. In some cases, sperm may be collected from urine. In severe cases of low or no sperm count, sperm or spermatids may be retrieved through the microsurgical procedures that include microsurgical sperm aspiration from the epididymis (MESA), percutaneous sperm aspiration from the epididymis (PESA), biopsy and sperm extraction from the testicle (TESE), and percutaneous sperm aspiration from the testicle (TESA). Male gametes may also be produced in vitro from the culture of testicular tissue and stem cells.

### III.C. Embryo Generation

In some cases, embryos may be generated through in vitro fertilization. In other cases, embryos produced through fertilization in vivo. In some cases, fertilization may be facilitated by intracytoplasmic sperm injection, which comprises injecting a single sperm or spermatid into an egg. In some cases, embryos will be produced by co-incubating multiple sperm or spermatids and one or more eggs for a defined time period in conditions that facilitate fertilization, often referred to as in vitro fertilization (IVF, see patents U.S. Pat. No. 6,610,543 and U.S. Pat. No. 6,130,086, incorporated by reference herein).

In some cases, zygote production may comprise nuclear transfer from a donor cell into an enucleated oocyte or zygote. A nucleus or pronuclei may be transferred from the donor cell. Fertilization may be monitored by noting the presence of 2 pronuclei within hours after fertilization and/or mitotic division within 24 hours following fertilization.

### III.D. Embryo Culture

After fertilization, embryos may be maintained in conditions that are optimal for development using known methods. Most commonly, embryos are maintained in small drops of specially designed culture medium on culture dishes that are overlaid with mineral or paraffin oil. These dishes are maintained in an incubator that provides an environment optimized for embryonic health and development. Typical conditions may include a temperature approximating that found in vivo (35-37° C.), sub-ambient concentration of oxygen (usually 5%) and elevated concentrations of CO₂ (5-6%). The developmental progression and potentially other physiologic parameters of the embryo are followed serially throughout the culture period. Mammalian embryos are typically maintained in culture for a period up to the length of the natural preimplantation period. For example, human embryos may be maintained in culture for up to 6 days. A number of other culture environments may be used in which a number of components or features of the system differ, including the volume of culture media, shape of the culture vessel, composition of vessel substrate, composition of culture medium, use of static or dynamic culture systems, mechanical or flow-induced movement of embryos, circulation or exchange of media type of incubator and physiologic monitoring systems. Embryos may be cryopreserved at any time point during this period using techniques that are known in the art.

### IV. Acquisition of RNA Samples from Embryos

At some stage of early embryonic development, the methods and compositions of this disclosure provide for acquisition of a sample containing RNA that is representative of all forms of RNA expressed from cells of the embryo. RNAs obtained from an embryo may include any RNA, including but not limited to mRNA, rRNA, tRNA, nRNA, siRNA, snRNA, snoRNA, scaRNA, microRNA, dsRNA, ribozymes and riboswitches.

There are two general approaches that may be used: invasive methods in which a sample is removed from the embryo and noninvasive methods in which cells or RNA that have been naturally released from the embryo are collected.

### IV.A. Invasive Methods for Obtaining an RNA Sample

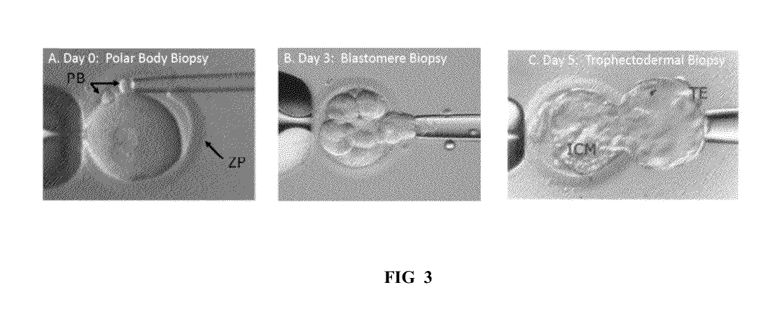

The methods and compositions of this disclosure provide for any method suitable for the acquisition of a sample representative of RNA through invasively sampling of the embryo. In some cases, a sample may be obtained by biopsying the embryo to remove one or more cells from the embryo using techniques known in the art (see Xu and Montag (2012) Seminars in Reproductive Medicine 30: 259-266, incorporated by reference herein). Examples of several common biopsy methods are shown in FIG. 3. In some cases of the compositions and methods of this disclosure, the embryo is biopsied at the blastocyst stage (FIG. 3.C). Biopsy at this stage involves the removal of trophectodermal cells that enclose the fluid-filled blastocoel and inner cell mass. In the case of humans, for example, a blastocyst is usually biopsied on day 5 or day 6 following fertilization (i.e., 120-144 hrs post fertilization) using standard methods, such as those described in McArthur, et al. (2008) Prenatal Diagnosis 28: 434-442, incorporated by reference herein. Generally, the trophectoderm is promoted to herniate out of the zona pellucida (ZP) through a breach created by a diode near-infrared laser such as the Octax or Fertilase (MTM), Saturn 5 (RI) or Zilos-tk (Hamilton Thorne) lasers. In other embodiments, this breach may be created through the use of other mechanical means (e.g., blade or needle), chemical means (e.g., acidic Tyrode's solution) or thermal means (e.g., direct contact with a heating element). In the case of human embryos, the ZP breach is generally performed on day 3 of 4 of culture. Blastocysts with herniation of the trophectoderm through the trophectoderm (FIG. 3.C) are ideal for biopsy. Blastocysts that have fully hatched from the zona pellucida and those that have not hatched at all may also be biopsied. In the case of fully enclosed blastocysts, it may be possible to use the breach previously placed in the zona pellucida or it may be necessary to enlarge this breach or make a new breach to obtain a sample. In other cases, the ZP is not breached until immediately prior to biopsy.

In the some cases, fresh blastocysts (embryos that have not been cryopreserved) are biopsied. In other cases, biopsies are performed on embryos generated from cryopreserved gametes or from embryos that have been previously cryopreserved.

During biopsy, blastocysts are placed in individual small drops of culture medium with oil overlays and are transferred to an inverted microscope with a heated stage. The embryo may be secured by gentle suction to a thick-walled, blunt-ended pipet, known in the art as a holding pipet. The holding pipette may be maneuvered using a micromanipulator. The herniation may be oriented toward the biopsy pipet and a smaller bore biopsy pipet may be generally used to attach and/or draw out a small portion of the herniation into the pipet's lumen using gentle suction. A near-infrared laser may be used to detach a small segment of the trophectoderm containing 1-20 cells using multiple low power laser pulses. In some cases, more than one biopsy may be performed.

Other methods may also be used to secure and manipulate the embryo. Alternative methods may include any application that uses suction or physical constraint to keep the embryo at a defined location. In some cases, optical tweezers could also be used to hold the embryo (see Ilina, (12) in International Symposium on High Power Laser Ablation, Phipps, C. Ed., pp. 560-571, incorporated by reference herein).

Other alternative methods may be used to release the biopsy sample from the embryo. In some cases, a biopsy sample may be torn, e.g., dragging the biopsy pipet across the face of the holding pipet. In other cases the biopsy may be cut from the embryo, e.g., using a blade or other cutting device (see Perez (12) Fert Steril 98: S140, incorporated by reference herein).

Further, chemical methods may be used to release the biopsy sample from the embryo. In some cases intercellular connections or bridging cells are disrupted by localized delivery of chemicals agents. Chemical agents may include but are not limited to detergents or hypotonic solutions, or enzymes such as trypsin or proteinase K. The methods and compositions of this disclosure provide for any suitable method or combination of methods to obtain the biopsy specimen.

Additionally, in some cases as provided by this disclosure, the embryo may be biopsied at an earlier or later stage during development than the blastocyst stage. For earlier stages, any stage may be analyzed that follows activation of at least some of the embryonic genome, which corresponds to between 24-48 hours after fertilization in human embryos. In some cases, the earlier stage may be the early cleavage stage in which there are 6-10 cells (FIG. 3.B). At this stage, which usually corresponds to the 3^{rd} day following fertilization, the embryo may be transferred to media containing no divalent cations and/or chelating agents to promote dissociation of the blastomeres. Using micromanipulator and laser equipment as described herein, the ZP is breached and 1 or 2 blastomeres may be removed using the biopsy pipet. In other cases, embryos may be split at the 2-8 cell stage (see Tang (12) Taiwanese J of Obstet Gyn S1: 236-9, incorporated by reference herein). In this case, one embryo may be sampled or used in its entirety for genomic analysis while the other may be reserved to establish a pregnancy if appropriate. In some cases, a system that is capable of simultaneously biopsying multiple embryos may be used.

In some cases, cells obtained for biopsy may be at least about 1, 2, 3, 4, 5, 6, 7, 8, 9, 10, 11, 12, 13, 14, 15, 16, 17, 18, 19, 20, 50, or 100 cells. In some cases, cells obtained for biopsy may comprise at most about 1, 2, 3, 4, 5, 6, 7, 8, 9, 10, 11, 12, 13, 14, 15, 16, 17, 18, 19, 20, 50, or 100 cells.

In some cases, the biopsy will be performed to remove one or more subcellular compartments of the cell rather than the intact cell. Subcellular compartments include, but are not limited to, the nucleus, mitochondria and cytoplasm. Such subcellular sampling may be performed using very fine gauge biopsy pipets with our without the aid of piezo.

In some cases, cells will be lysed in situ and the lysate containing RNA may be obtained immediately following lysis. In this method, a lysis method as described below may be delivered locally to lyse one or more embryonic cells. The lysed cellular content may then be immediately retrieved through aspiration.

In some cases, cells will be lysed in situ and the lysate containing RNA will be obtained during the biopsy process.

In other cases, one or more subcellular compartments of the cell will be obtained during biopsy. Subcellular compartments include, but are not limited to, the nucleus, mitochondria and cytoplasm.

In some cases, lysates or subcellular components may be obtained from least about 1, 2, 3, 4, 5, 6, 7, 8, 9, 10, 11, 12, 13, 14, 15, 16, 17, 18, 19, 20, 50, or 100 cells. In some cases, lysates or subcellular components may be obtained from least about 1, 2, 3, 4, 5, 6, 7, 8, 9, 10, 11, 12, 13, 14, 15, 16, 17, 18, 19, 20, 50, or 100 cells.

### IV.B. Noninvasive Acquisition of RNA Samples

In some cases, embryonic cells may be obtained without a biopsy procedure through the collection of cells that have been released from the embryo. These cells may be collected from the culture medium or by collecting cells that are contained within or adherent to the zona pellucida (ZP) following removal and/or collection of the ZP.

Further, a sample of cell-free RNA released from an embryo may also be obtained noninvasively for the compositions and methods of this disclosure. In some cases, cell free RNA may be obtained from the embryo culture medium (see Rosenbluth, (12) Fert Steril 98: S19, incorporated by reference herein). In other cases, it may be possible to isolate RNA that is contained within or adherent to the ZP following removal and/or collection of the ZP. In other cases, RNA may be obtained from both culture medium and the ZP.

In other cases, embryonic cell free RNA may be isolated from bodily fluids of a mother including but not limited to blood, serum, plasma, genital tract secretions or washings, vitreous, sputum, urine, tears, perspiration, saliva, mucosal excretions, mucus, spinal fluid, lymph fluid and the like.

Isolation and extraction of cell free RNA may be performed through a variety of techniques. In some cases, collection may comprise aspiration of a fluid from a subject using a syringe. In other cases collection may comprise pipetting or direct collection of fluid, i.e. culture media, from a vessel or droplet.

### IV.C. Timing of Sample Acquisition

In some cases, invasive or noninvasive samples may be obtained at least about 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or, 3 weeks after fertilization of the embryo. In some cases, cells obtained for biopsy of an embryo may comprise at most about 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or 3 weeks after fertilization of the embryo.

In some cases, invasive or noninvasive samples may be obtained at least about 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or, 3 weeks after expression of the embryonic genome. In some cases, cells obtained for biopsy of an embryo may comprise at most about 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or 3 weeks after expression of the embryonic genome.

### V. Sample Preparation and Generation of Raw Transcriptome Data

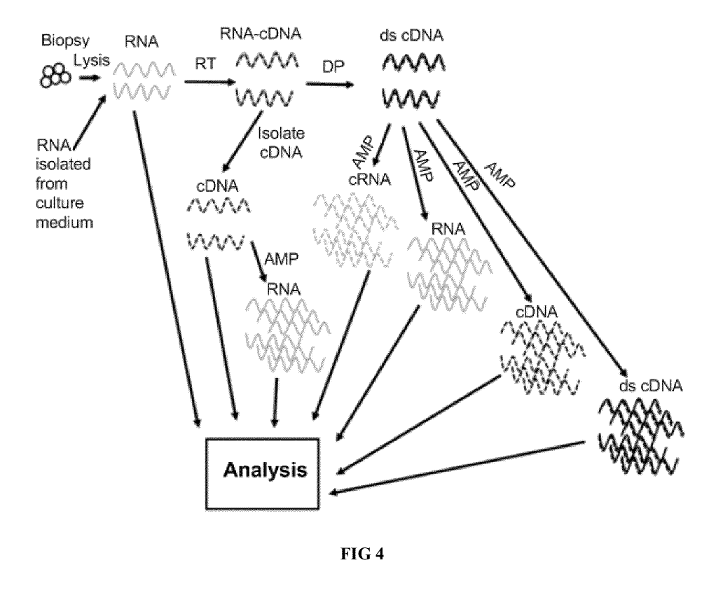

Generally, any suitable method that can be used to identify and quantitate the expression levels of multiple transcripts simultaneously from the types of samples described above may be used for this application. In most cases, it is advantageous to use a method that can evaluate all or a large percentage of transcripts in the sample to increase the coverage, resolution and sensitivity of the method. Furthermore, a more comprehensive assessment of the transcriptome opens up the possibilities for incorporation of a number of other biological evaluations. Methods that allow for such analysis include but are not limited to massively parallel sequencing (RNA-Seq) and multiplexed hybridization-based or amplification-based methods of transcriptome profiling. The advantages of RNA-Seq relative to these other methods include unbiased analysis, large dynamic range and high throughput. A variety of nucleic acids may be generated and used for transcriptome analysis (FIG. 4). Since most of the currently available methods require lysis of cell samples, RNA isolation, cDNA generation and nucleic acid amplification, these steps will be first presented in a general manner with notes pertaining to the particular downstream application. Following these sections, a section will follow describing the steps that are more specific for each of the three classes of analytic approaches: RNA-Seq, hybridization-based and amplification-based methods. Those skilled in the art will appreciate that these sections are only exemplary, and a number of different approaches could be employed to achieve similar results.

### V.A. Cell Treatment and Lysis

In cases in which a sample containing cells is obtained, it will be necessary to lyse cells to release the RNA. In cases in which cell-free RNA or a lysate is obtained, this step would not be necessary. Compositions and methods of this disclosure provide for any suitable methods for preparing cell samples for processing for transcriptome analyses. In some cases, the entire cell sample may be immediately processed for downstream analysis. In other cases, the cell sample is processed in a number of ways before proceeding with the steps for molecular diagnostics. In some cases, the cell sample is divided, or cells are dissociated so that more than one sample may be derived from the biopsy. In other cases, the cells may be cultured so that more cellular material may be available for analysis. Further, the entire or a portion of the biopsy sample may be cryopreserved so that the cells can be revived and cultured at a later timepoint.

In some cases, the sample of cells may be treated to facilitate the isolation of specific subspecies of RNAs using cross linking agents such as ultraviolet light or chemicals. In other cases, samples may be exposed to BrdU to facilitate isolation of recently synthesized RNA.

In some methods, the cell samples are washed one or more times in a solution to remove components from the culture or biopsy medium and any extraneous nucleic acids. Any solution that is devoid of nucleases and extraneous nucleic acids, that does not stress the cells and that facilitates handling of samples may be used. A typical solution is phosphate-buffered saline containing 5 mg/ml molecular biology grade bovine serum albumin, but alternative solutions may be used. Samples may be washed by transferring samples to several drops of wash solution under oil using pipettes that have an inner diameter that is close to the size of the biopsy sample (generally in the 1-5 micron range) and drawing the sample in and out of the pipet several times. Alternative means of exposing the sample to wash solution may be used.

In cases in which a sample from an embryo comprises cells, the cells must be lysed to release RNA. In some cases, cells may be lysed in a hypotonic solution containing a weak detergent, RNAse inhibitors and a sufficiently large volume to substantially dilute cellular constituents. One such protocol is to place the biopsy sample in hypotonic lysis buffer consisting of 0.2% Triton X-100 and RNase inhibitors in RNase free water. Any solution that facilitates lysis and allows for downstream processing and analyses may be used. Lysates may then be either frozen or immediately processed for transcriptome analysis. Samples to be frozen may be rapidly cooled by submerging the tube in liquid nitrogen and then storing at −80 C or colder temperatures until subsequent processing.

In other cases, alternative methods may be used to lyse cells (see Brown and Audet (2008) Journal of The Royal Society Interface 5: S131-S138, incorporated by reference herein). Methods may include but are not limited to the use of various hypotonic solutions, other or differing concentrations of detergents (e.g. SDS, NP40), low or high pH, other lysis-inducing chemicals (e.g. chaotropic salts such as guanidinium isothiocyanate), enzymes (e.g., proteinase K), freeze-thaw cycles, heat (e.g., exogeneous heat from a conductor, heated solution or laser), mechanical disruption (e.g., contact with sharp object or sonication) or electroporation or any combination of the aforementioned approaches. Kits such as CellsDirect (Invitrogen) and Cells-to-CT (Applied Biosystems) may also be used with the compositions and methods of this disclosure. Any method that can effectively lyse the cells and allow for subsequent processing and analytic steps may be used.

### V.B. RNA Purification and Preparation

In some instances, the cell lysate or obtained RNA sample may be used directly for sequencing or subsequent processing steps. In other instances, total RNA or subclasses of RNA may be isolated before sequencing or processing. The compositions and methods of the disclosure provide for any suitable methods of RNA isolation and purification that are compatible with subsequent transcriptome analysis.

Any commercially available method for purifying total RNA from a small number of cells that is compatible with downstream transcriptome analyses may be used. In some cases, RNA may be isolated using commercially available kits such as those provided by companies such as Arcturus, Sigma Aldrich, Life Technologies, Promega, Affymetrix, IBI or the like. Kits and protocols may also be non-commercially available. In some cases methods may use a silica-gel membrane.

In other compositions and methods of this disclosure, a subset of species of RNA may be isolated or selected for subsequent processing. Since ribosomal RNAs (rRNA) constitute >80% of transcripts within cells, some methods may take steps to reduce the amount of these sequences present in the sample. In some cases, hybridization methods may be used either to deplete rRNA sequences or to select for polyadenylated RNA, which mainly consists of messenger RNA (mRNA). In some cases, rRNA may be depleted by hybridization with biotin labeled oligonucleotide probes and subsequent removal using streptavidin-coated magnetic beads as provided by commercially available kits such as RiboMinus kit (Invitrogen) or Ribo-Zero (Epicentre). In other cases, polyadenylated RNA may be selected using oligo-dT probes linked to substrates or beads. In other cases, rRNA may be removed through selective degradation. Since rRNA has exposed 5′ phosphates (in contrast to mRNA that has a capped 5′ end), rRNA molecules may also be removed by using an exonuclease able to specifically degrade RNA molecules bearing a 5′ phosphate such as provided by the mRNA ONLY kit (Epicentre). rRNA may also be degraded using cDNAs complementary to rRNAs and a duplex-specific nuclease (DSN).

In other cases, select sequences within the transcriptome may be enriched through the use of the targeted capture techniques. In some cases, this targeted capture technique may comprise incubating the lysate with primers of target sequences that are immobilized to a substrate, washing away unbound RNA and then retrieving target sequences. Target capture of RNA sequences may be performed using a number of commercially available kits including, but not limited to, Agilent's SureSelect system and Illumina's TruSeq system.

In other cases, immunoprecipitation may also be used to isolate RNAs that have been cross-linked to specific proteins using methods described above (see Churchman and Weissman (2011) Nature 469: 368-375; Ingolia, et al. (2009) Science 324: 218-223; Licatalosi, et al. (2008) Nature 456: 464-470, incorporated by reference herein).

In some cases, intact RNA may be used for subsequent steps. In other cases RNA may be fragmented prior to subsequent processing. RNA may be fragmented by any appropriate means including, but not limited to, elevated temperature, exposure to chemicals (e.g. metal ions), exposure to enzymes (e.g. RNases) or nebulization. RNA fragmentation may eliminate some of the challenges associated with RNA secondary structure.

In some cases, adapters may be ligated to the RNA prior to subsequent processing. These adaptors may facilitate reverse transcription, tagging, amplification and/or purification.

In some cases, exogenous RNAs not present in the sample may be added to the lysate or isolated RNA sample. These spike in RNAs may improve quantitation by allowing for the efficiency of the subsequent processing steps to be assessed.

### V.C. Reverse Transcription

For some analytic approaches, RNA is converted into cDNA using reverse transcriptase in any suitable method. Various techniques for reverse transcription are known in the art. Reverse transcription of mRNA may be primed with the use of specific primers, such as oligo-dT and/or random primers.

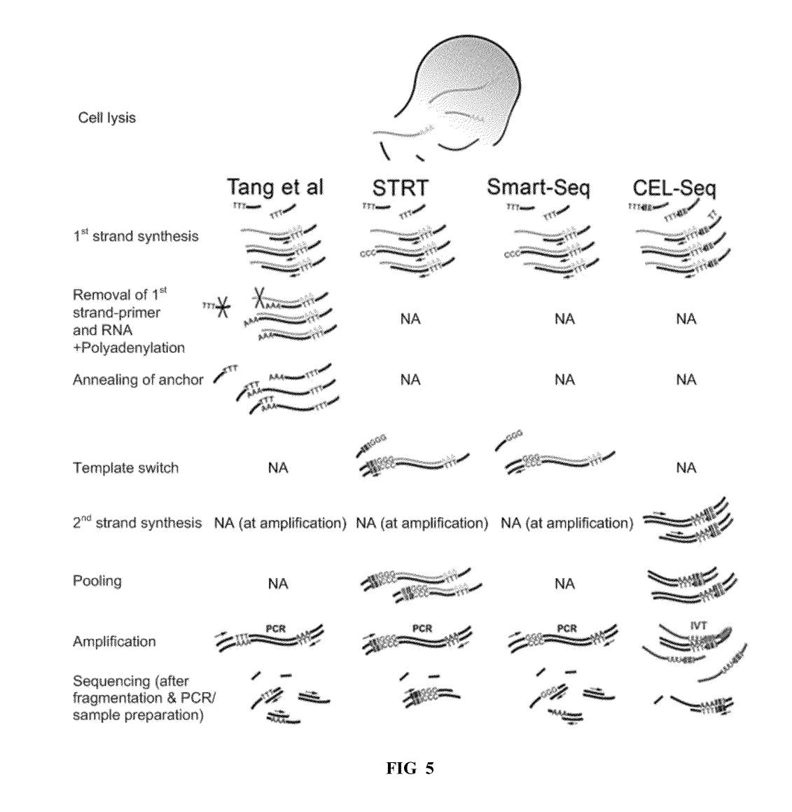

In some composition and methods of this disclosure, both the first and second strands of cDNA are synthesized simultaneously using a template strand switching technique by adding a reaction mix directly to the sample lysate (see Zhu, et al. Biotechniques 30: 892-897, incorporated by reference herein). An oligodT primer may be used by Moloney murine leukemia virus (MMLV) reverse transcriptase to reverse transcribe the first strand. Following completion of the reverse transcription, a polycytosine tract is added to the strand due to MMLV's terminal transferase activity. Inclusion of a primer with a sequence that is complementary to the polyC tract, allows extension of the second strand. This technique generally referred to as switch mechanism at the 5′ end of RNA templates (SMART) and may be provided by such as the Clontech SMARTer™ Ultra Low RNA Kit (FIG. 5). In alternative composition and methods, different primers and reverse transcriptases may be used to produce double stranded cDNA by template switching (FIG. 5).

Double-stranded cDNA may also be produced using a protocol that uses a reverse transcriptase without terminal transferase activity. A poly(dT)-tailed primer is first used to reverse transcribe RNA. The unpolymerized primer is degraded with exonuclease and the cDNA is polyadenylated with terminal transferase. A poly (dT) primer is then used to complete the second strand synthesis using DNA polymerase I.

In other methods, primers with unique identifiers, or bar codes, may be used in the reverse transcription and/or second strand synthesis steps that allow for quantitation. Bar codes may be used to identify the source of RNA, or used as a tool to count or quantify transcripts as described herein (see Kivioja, et al. (2012) Nat Methods 9: 72-83; Shiroguchi, et al. (2012) Proc Natl Acad Sci USA 109: 1347-52, incorporated by reference herein).

In other applications, cDNA may be synthesized by ligating adapters to the RNAs to serve as primer annealing sites. Random primers can also be used to prime the reverse transcription throughout the RNA. In other applications, the primer mix may be semi-random with primers binding to certain sequences such as rRNAs being omitted.

In some cases, alternative methods may be used to preserve strand information such that it will be possible to determine which strand of DNA was transcribed to generate the transcript of interest. Directional, strand-specific information may be used for comprehensive annotation of the transcriptome and for identifying antisense transcription. In some cases, different adaptors sequences are attached in a known orientation relative to the 5′ and 3′ ends of the RNA transcript. These protocols generate a cDNA library flanked by two distinct adaptor sequences, marking the 5′ end and the 3′ end of the original mRNA. In other cases, one strand may be marked by chemical modification, either on the RNA itself by bisulfite treatment or during second-strand cDNA synthesis followed by degradation of the unmarked strand (as described by Levin, et al. (2010) Nat Methods 7: 709-715, incorporated by reference herein).

In other applications, only a single-stranded cDNA may be synthesized as a substrate for amplification. In the case of in vitro transcription (iVT) based amplification methods, specific binding and initiation sites may be introduced such as 5′ extensions corresponding to one of the phage RNA polymerase priming and recognition sites (see application U.S. Pat. No. 5,514,545A, incorporated by reference herein). In some cases, a polynucleotide tract may be added to the cDNA to facilitate PCR-based amplification. In some cases the cDNA may be fragmented or digested to allow for sequencing of one end of the cDNA (Hashimshony, et al. (2012) Cell Reports 2: 666-673; Islam, et al. (2012) Nat Protoc 7: 813-828, incorporated by reference herein).

In some cases, reverse transcription reaction may be used to directly sequence RNAs using single molecule sequencing such as the Helicos system as described by Ozsolak and Milos (2011) Wiley Interdisciplinary Reviews-Rna 2: 565-570, incorporated by reference herein. Other systems capable of single molecule sequencing system could be modified to sequence unamplified RNA, including the single molecule sequencing system of Pacific Biosciences and the system being developed by Oxford Nanopore Technologies.

In some cases, reverse transcription reaction may be used to generate one of more copy of each cDNAs that may then be sequenced. In one example of the technique, referred to as on-flow cell reverse transcription sequencing (FRT-Seq), fragmented and adaptor ligated RNA is placed in an Illumina flow cell containing appropriate bound primers and reverse transcriptase to generate clusters of cDNAs by bridging amplification (as described by Mamanova and Turner (2011) Nat Protoc 6: 1736-47, incorporated by reference herein).

In some cases, the cDNA is sequenced rather than the RNA. Any of the methods described herein for single molecule sequencing as described above could be used for this sequencing. Many of the single molecule sequencing systems available or being developed by Helicos, Pacific Biosciences and Oxford Nanopore technologies are developed specifically for sequencing DNA molecules.

V.D. cDNA Amplification

Most currently available methods for transcriptome profiling require more input nucleic acid than would be present in samples obtained from embryos. Consequently, the RNA or cDNA generated from such samples must be amplified. Compositions and methods of this disclosure provide for any suitable methods for the amplification of products of reverse transcription, (see FIG. 4). In some cases, cDNA molecules contain sequences at each end of the cDNA that serve as priming sites for amplification by PCR as shown in FIG. 5. PCR-based amplification may be performed using any suitable method known in the art (U.S. Pat. Nos. 4,683,195; and 4,683,202; PCR Technology: Principles and Applications for DNA Amplification, ed. H. A. Erlich, Freeman Press, NY, N.Y., 1992).

In some cases, all cDNAs are amplified. In other cases, only a subset of cDNAs is amplified. In some cases, the subset is randomly selected. In other cases, the cDNAs for amplification are specifically selected.

In some cases, a thermoresistant polymerase with high processivity such as the Advantage 2 Polymerase (Clontech) may be used to enhance the amplification of entire transcripts (see Ramskold, et al. (2012) Nat Biotechnol 30: 777-82, incorporated by reference herein).

Suitable alternative methods may use different primers, thermoresistant polymerases and/or amplification solutions (buffer, dNTPs, and additional reagents that may improve the amplification reaction). For example, evaluation of gene expression involving amplification of the 5′ fragments of cDNAs using universal primers may be performed as described by Islam Islam, et al. (2012) Nat Protoc 7: 813-828, incorporated by reference herein. In some cases, quasi-linear preamplification referred to as multiple annealing and looping-based amplification cycles (MALBAC) may also be applied to amplifying cDNAs (as described by Zong, et al. (2012) Science 338: 1622-6, incorporated by reference herein).

In addition to PCR, compositions and methods of this disclosure may use any other method for amplifying nucleic acids to amplify transcribed sequences present in embryo biopsy samples (for review of amplification techniques (see Wang, et al. (2009) Nat Rev Genet 10: 57-63 and Nygaard and Hovig (2006) Nucleic Acids Research 34: 996-1014, incorporated by reference herein).

In other cases of amplifying cDNA sequences, a linear method of amplification such as in vitro transcription and single primer isothermal amplification (SPIA)(Kurn, et al. (2005) Clin Chem 51: 1973-81 and Nugen U.S. Pat. Nos. 6,692,918; 6,251,639; 6,946,251 and 7,354,717, incorporated by reference herein) have been used for amplifying cDNAs from single or small numbers of cells. Methods that combine both in vitro transcription and PCR have also been developed such as the CEL-Seq method developed by Hashimshony, et al. (2012) Cell Reports 2: 666-673, incorporated by reference herein. In this method, adapters are ligated to 5′ end of in vitro transcribed RNAs, the RNAs are fragmented and another adapter is added to the 3′ end. Those fragments containing both adapters, representing the 5′ end of RNAs, are then amplified by PCR. Since this method ligates 2 different adapters, it will also be possible to determine the strandedness of the RNA that produced the clone.

Those skilled in the art will recognize that many additional methods of nucleic acid amplification could be used, including but are not limited to, polymerase chain reaction (PCR), ligase chain reaction (LCR) (Wu and Wallace, Genomics 4:560, 1989; Landegren et al., Science 241:1077, 1988, incorporated by reference herein), strand displacement amplification (SDA) (U.S. Pat. Nos. 5,270,184; and 5,422,252, incorporated herein by reference), transcription-mediated amplification (TMA) (U.S. Pat. No. 5,399,491, incorporated herein by reference), linked linear amplification (LLA) (U.S. Pat. No. 6,027,923, incorporated herein by reference), self-sustained sequence replication (Guatelli et al., Proc. Nat. Acad. Sci. USA, 87, 1874 (1990) and WO90/06995, incorporated herein by reference), selective amplification of target polynucleotide sequences (U.S. Pat. No. 6,410,276, incorporated herein by reference), consensus sequence primed polymerase chain reaction (CP-PCR) (U.S. Pat. No. 4,437,975, incorporated herein by reference), arbitrarily primed polymerase chain reaction (AP-PCR) (U.S. Pat. Nos. 5,413,909, 5,861,245, incorporated herein by reference) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al., (92), Nucleic Acids Res. 20(7):1691-6, incorporated herein by reference, rolling circle amplification, described in U.S. Pat. No. 5,648,245, incorporated herein by reference, and balanced PCR (Makrigiorgos, et al. (02)). Nature Biotechnol, 20:936-9 (2002)). Other amplification methods that may be used are described in U.S. Pat. Nos. 5,242,794; 5,494,810; 4,988,617, U.S. Ser. No. 09/854,317 and US Pub. No. 20030143599, each of which is incorporated herein by reference. In some aspects DNA is amplified by multiplex locus-specific PCR. Based on such methodologies, a person skilled in the art readily can design primers in any suitable regions 5′ and 3′ to a locus of interest and amplify segments or complete cDNA sequences of transcripts.

In another approach, a subset of amplified cDNAs may be selected following amplification using various hybridization-based target sequence capture as described herein.

In cases in which the amplified nucleic acids will be quantitated by hybridization-based methods, amplification products may be labeled through the use of nucleotides that are conjugated to labels. Labels may be any molecule or compound that can be attached to one or more nucleotides and both permit incorporation of the nucleotide into the amplification product and facilitate detection of the amplification product. Such labels may include fluorophores, chemiluminescent agents, enzymes or radioactive molecules. Alternatively, nucleotides may be linked to molecules that allow for indirect detection following binding of a secondary labeled molecule. Indirect labeling methods include, but are not limited to, biotin-streptavidin and antigen-antibody systems. The choice of label may depend on sensitivity required, ease of conjugation with the probe, stability requirements, and available instrumentation. Alternatively, the amplification products may be labeled following the amplification procedure.

In cases in which the amplified nucleic acids will be quantitated by amplification-based methods, the initial amplification of the cDNA, often referred to as a preamplification, may be restricted to amplifying only a subset of sequences (i.e., sequences that will be assayed) and the degree of amplification may be smaller, such that a limited number of amplification products are initially produced. This may be achieved through various methods, such as limiting PCR amplification cycles or the use of linear amplification techniques. This preamplification may be used to generate sufficient numbers of templates to allow for numerous amplification-based assays to be run in parallel. In various embodiments employing preamplification, the preamplification may also be used to add one or more nucleotide tags to the target nucleotide sequences so that the relative copy numbers of the tagged target nucleotide sequences is substantially representative of the relative copy numbers of the target nucleic acids in the sample. Preamplification may be carried out for 2-20 cycles to introduce the sample-specific or set-specific nucleotide tags. In some cases, the annealing sequences of the primers used for preamplification may be the same as is used in the subsequent quantitative assays. In other cases, primers that bind to sequences distal to the primer binding sites for the quantitative assay may be used in a ‘nested’ amplification strategy.

Amplification of the cDNA may yield RNA (same strand as the original RNAs in the sample), complementary RNA, single stranded cDNA, single-stranded DNA from the coding strand or double-stranded cDNA (FIG. 4).

After production of sufficient nucleic acids derived from the sample RNA by one of the amplification methods described herein, the amplified nucleic acids may be analyzed using one of several high throughput methods to generate data that can be used to evaluate the expression of that include massively parallel sequencing, multiplexed hybridization to probes or multiplexed amplification-based assays.

### V.E. Sample Preparation and Raw Data Generation for Sequencing-Based Transcriptome Profiling

After production of larger amounts of nucleic acids derived from the sample RNA by amplification, compositions and methods of the disclosure provide for subsequent sequencing of these nucleic acids. For a number of currently available massively parallel sequencing technologies, such as the HiSeq/MiSeq (Illumina), SoLiD/Ion Torrent (Life Technologies), 454 GS FLX+/GS Junior (Roche), and Complete Genomics platforms, libraries are generated to facilitate sequencing. Sequencing libraries consist of clones containing inserts of short fragments of DNA flanked by sequences that may be used to sequence one or both ends of the insert DNA. The protocols for preparation of libraries are specific to each sequencing platform, although the principle steps are similar, involving fragmentation of input DNA, ligation of adaptors, multiplexed amplification of individual clones and sequencing of amplified clones in parallel. An overview of the steps required for library preparation is described herein. Those skilled in the art will recognize the appropriate steps required for preparing a suitable library for a particular downstream sequencing platform. Detailed protocols and descriptions of kits for preparing libraries for specific platforms can be obtained at the manufacturer's websites: HiSeq/MiSeq (www.Illumina.com) SOLiD/Ion Torrent (www.lifetechnologies.com) and 454 Sequencing (www.454.com). Since the Complete Genomics library preparation is provided as part of their service, the methods for this approach are not considered in detail. With any of these protocols, a number of modifications can be used to improve the process or tailor it for a specific sample type or even modify for a different library-based sequencing platform.

V.E.i. DNA Fragmentation

In some cases of sequencing, such as with most currently available massively parallel sequencing technologies, nucleic acids may need to be reduced to fairly small fragments to increase coverage from the relatively short sequence reads from the end terminus/termini of the nucleic acids.

In some cases, cDNAs may be fragmented into sizes of at least 10, 20, 30, 40, 50, 60, 70, 80, 90, 100, 200, 300, 400, 500, 600, 700, 800, 900, 1000, 2000, 3000, 5000 base pairs in length. In some cases cDNAs may be fragmented into sizes of at most 10, 20, 30, 40, 50, 60, 70, 80, 90, 100, 200, 300, 400, 500, 600, 700, 800, 900, 1000, 2000, 3000, 5000 base pairs in length.

Numerous fragmentation methods are described herein and known in the art. For example, fragmentation may be performed through physical, mechanical or enzymatic methods. Physical fragmentation may include exposing a target polynucleotide to heat or to UV light. Mechanical disruption may be used to mechanically shear a target polynucleotide into fragments of the desired range. Mechanical shearing may be accomplished through a number of methods known in the art, including repetitive pipetting of the target polynucleotide, sonication and nebulization. Target polynucleotides may also be fragmented using enzymatic methods. In some cases, enzymatic digestion may be performed using enzymes such as using restriction enzymes.

Restriction enzymes may be used to perform specific or non-specific fragmentation of target polynucleotides. The methods of the present disclosure may use one or more types of restriction enzymes, generally described as Type I enzymes, Type II enzymes, and/or Type III enzymes. Type II and Type III enzymes are generally commercially available and well known in the art. Type II and Type III enzymes recognize specific sequences of nucleotide base pairs within a double stranded polynucleotide sequence (a “recognition sequence” or “recognition site”). Upon binding and recognition of these sequences, Type II and Type III enzymes cleave the polynucleotide sequence. In some cases, cleavage will result in a polynucleotide fragment with a portion of overhanging single stranded DNA, called a “sticky end.” In other cases, cleavage will not result in a fragment with an overhang, creating a “blunt end.” The methods of the present disclosure may comprise use of restriction enzymes that generate either sticky ends or blunt ends.

Restriction enzymes may recognize a variety of recognition sites in the target polynucleotide. Some restriction enzymes (“exact cutters”) recognize only a single recognition site (e.g., GAATTC). Other restriction enzymes are more promiscuous, and recognize more than one recognition site, or a variety of recognition sites. Some enzymes cut at a single position within the recognition site, while others may cut at multiple positions. Some enzymes cut at the same position within the recognition site, while others cut at variable positions.

In some cases, Nextera kits such as provided by Illumina/Epicentre, which use a tn5 transposase to simultaneously fragment the double-stranded DNA and ligate sequencing platform specific adaptors to the ends of the fragments, may be used. Alternative kits such as MuSeek (Life Technologies), or other fragmentation/tag techniques may be used.

In some cases, cDNA fragmentation may not be performed. Rather, RNA molecules, before reverse transcription to cDNA, may be fragmented using any suitable method as described herein.

In some cases, the fragmented DNA is size-selected using agarose gel methods such as SizeSelect™ Gels (Life Technologies) or Pippin Prep™ kits or beads such as AMPure XP (Beckman Coulter). In other embodiments, fragmented DNA is end repaired or polynucleotide tailed for subsequent steps of library preparation.

V.E.ii. DNA Strand End Repair

In many cases, fragmentation of DNA, such as through mechanical shearing or enzymatic digestion, results in fragments with a heterogeneous mix of blunt and 3′- and 5′-overhanging ends. In some cases, the compositions and methods of the disclosure provide for repair of the fragment ends using methods or kits (i.e. Lucigen DNA terminator End Repair Kit) known in the art to generate ends that are designed for insertion, for example, into blunt sites of cloning vectors. In some cases, the compositions and methods of the disclosure provide for blunt ended fragment ends of the population of DNAs sequenced. Further, in some cases, the blunt ended fragment may also be phosphorylated. The phosphate moiety can be introduced via enzymatic treatment, for example, using a kinase, (i.e. shrimp alkaline kinase).

In other cases, polynucleotide sequences are prepared with single overhanging nucleotides by, for example, activity of certain types of DNA polymerase such as Taq polymerase or Klenow exo minus polymerase which has a nontemplate-dependent terminal transferase activity that adds a single deoxynucleotide, for example, deoxyadenosine (A) to the 3′ ends of, for example, PCR products. Such enzymes can be utilized to add a single nucleotide ‘A’ to the blunt ended 3′ terminus of each strand of the target polynucleotide duplexes. Thus, an ‘A’ could be added to the 3′ terminus of each end repaired duplex strand of the target polynucleotide duplex by reaction with Taq or Klenow exo minus polymerase, whilst the adaptor polynucleotide construct could be a T-construct with a compatible ‘T’ overhang present on the 3′ terminus of each duplex region of the adaptor construct. This end modification also prevents self-ligation of both adapter and target such that there is a bias towards formation of the combined ligated adaptor-target sequences.

V.E.iii. Library Production and Amplification

For cases in which DNA has been fragmented and one of the currently available massively parallel sequencing platforms is used, platform specific protocols are used to prepare libraries of clones containing the fragmented DNA.

In some cases, a library may be prepared for an Illumina platform, comprising limited-cycle PCR in which a four-primer reaction adds bridge PCR (bPCR)-compatible adaptors to the core library (used for binding fragments to the flow cell). By including different Illumina compatible bar codes between the downstream bPCR adaptor and the core sequencing library adaptor in sets of 4 samples, 12 samples may be run on the same flow cell. Once the library is produced, size selected and quality confirmed, combinations of 12 samples with appropriate barcodes (12-plex/flow cell) are added to flow cells for cluster formation using the cBot. In this process, single molecules from the library bind to one of two oligonucleotides complementary to the different adapter sequences on the flow cell surface. Through repeated annealing and extension reactions of bridged sequences, clusters of around 1000 copies of the original library molecule may be formed on the flow cell substrate (Illumina (10) Technology Spotlight: Illumina Sequencing). In some cases there may be one or more clean-up steps to remove unligated adapters.

In other cases, library production and amplification may utilize the ligation of different adapters and PCR amplification under different conditions to generate a library for sequencing on other platforms. For example, individual library clones (single DNA molecules) may be bound to beads and each bead may be encapsulated in an aqueous droplet of PCR-reaction-mixture in oil, also known as emulsion PCR. The amplicons produced are also bound to the bead, thereby greatly increasing the number of copies bound to each bead. Such methods may be provided commercially, such as methods and kits sold by 454/Roche and SOLiD/Applied Biosystems. The primers used for the adaptors and sequencing are specific to each sequencing platform.

V.E.iv. Automation of Library Preparation

A number of solutions known in the art may be used to automate preparation of libraries suitable for the compositions and methods of this disclosure. For example microfluidic workstations, as provided by Fluidigm may aid in automation of workflow as described in Example 2. For example, the Fluidigm C1 workstation may be used with a biopsy sample as starting material and aid in and outputting libraries ready for sequencing on the the Illumina platform. Alternatively, kits and microfluidic systems, such as provided by Nugen (Mondrian: http://www.nugeninc.com/nugen/index.cfm/products/msp/tech/) may also be used.

V.E.v. Sequencing

Numerous methods of sequence determination are compatible with the assay systems of the disclosures. Exemplary methods for sequence determination include, but are not limited to, including, but not limited to, hybridization-based methods, such as disclosed in Drmanac, U.S. Pat. Nos. 6,864,052; 6,309,824; and 6,401,267; and Drmanac et al, U.S. patent publication 2005/0191656, which are incorporated by reference, sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, Proc. Natl. Acad. Sci., 100: 414-419 (2003); pyrophosphate sequencing as described in Ronaghi et al., U.S. Pat. Nos. 7,648,824, 7,459,311, 6,828,100, and 6,210,891; and ligation-based sequencing determination methods, e.g., Drmanac et al., U.S. Pat. Appin No. 20100105052, and Church et al, U.S. Pat. Appin Nos. 20070207482 and 20090018024.

Sequence information may be determined using methods that determine many (typically thousands to billions) of nucleic acid sequences in an intrinsically parallel manner, where many sequences are read out preferably in parallel using a high throughput process. Such methods include but are not limited to pyrosequencing (for example, as commercialized by 454 Life Sciences, Inc., Branford, Conn.); sequencing by ligation (for example, as commercialized in the SOLiD™ technology, Life Technology, Inc., Carlsbad, Calif.); sequencing by synthesis using modified nucleotides (such as commercialized in TruSeq™ and HiSeq™ technology by Illumina, Inc., San Diego, Calif., HeliScope™ by Helicos Biosciences Corporation, Cambridge, Mass., and PacBio RS by Pacific Biosciences of California, Inc., Menlo Park, Calif.), sequencing by ion detection technologies (Ion Torrent, Inc., South San Francisco, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

The amount of raw sequence data that is obtained for each sample is determined by the number of clones sequenced, whether one or both ends of clones are sequenced, and the length of sequence reads. The amount of sequence data will in turn impact the resolution of this approach for detecting CNVs. In some cases, only single end sequencing will be performed. In other cases, paired-end sequencing will be performed. The length of sequence reads may be more than 50, 100, 200, 300, 400, 500, 1000, 2000, 5,000 or 10,000 basepairs. The number of clones sequenced may be more than 1, 2, 5, 10, 20, 30, 40, 50, 60, 70, 80, 100 million.

### V.F. Sample Preparation and Raw Data Generation for Hybridization-Based Transcriptome Profiling

In some cases, RNA, cDNA, or amplified nucleic acids (i.e., RNA, cRNA, ss DNA, ss cDNA, ds cDNA) may be analyzed using hybridization-based methods. The basic principle for these methods is that labeled cDNAs are hybridized with probes using stringent conditions that favor highly specific annealing, i.e., favoring perfect or close to perfect matches. Following hybridization, the probes are washed under stringent conditions to remove unannealed and poorly annealed target sequences, and then target sequences that remain annealed are detected.

V.F.i. Expression Arrays

The most common embodiment for high throughput, hybridization-based transcriptome profiling at present is microarrays. There are several studies showing a high correlation between RNA-seq and expression microarray analysis results. Furthermore, we have found a very high correlation between RNA-seq and expression arrays for total RNA isolated from small pools of mouse embryos. For microarray analysis, RNA is isolated and amplified using the same general approaches as described for RNA-Seq with the exception that the amplified nucleic acids are labeled to facilitate detection. The nucleic acids may be labeled during or after the amplification process. There are several commercially available kits that perform both cDNA amplification and labeling of products: Ovation (Nugen), Message Amp (Ambion), Small sample target labeling (Affymetrix) and Bioarray small sample amplification (Enzo). In some embodiments, nucleic acid from another sample with a known genotype will be labeled with a different label so that the two samples can be competitively hybridized to allow for direct comparisons of expression between the 2 samples on 2-channel array platforms. The reference sample may be derived from one or more cells or embryos with defined genotype(s).

Following amplification, the nucleic acid is hybridized to a microarray. Expression microarrays contain thousands of probes that are complementary to known transcribed sequences that have been adhered to a substrate at defined locations. Microarrays may be printed, in situ-synthesized, high density bead and electronic and suspension bead microarrays. Arrays used may contain probes that detect all or a subset of transcripts from a sample. Microarrays may also be designed to assay allele-specific expression of loci through the use of probes specific for alleles of single nucleotide polymorphisms (SNPs) that correspond to different alleles of the loci. Microarray platforms used may be from commercial sources such as Affymetrix, Illumina, Roche NimbleGen and Agilent. Custom made arrays that contain user defined probes may also be used. In some instances such as the Illumina and Affymetrix platforms, only the amplified, labeled sample nucleic acid is hybridized to the array whereas with other platforms such as Roche NimbleGen and Agilent, the sample is cohybridized with a reference sample that contains a label that is distinct from that which is used to label the test sample. Conditions for hybridizations are well established for each platform type and should be familiar to those skilled in the art. Following hybridization, the microarrays are washed and scanned and the intensity values for all probes are recorded, also according to known protocols. The raw data from the scanned microarrays are measurements of signal intensities for the arrayed probes.

V.F.ii. Other Hybridization-Based Methods

In other embodiments, the hybridization of probe and targets are performed in solution rather than on an array. In general, all of these approaches perform a hybridization between probe and target sequences in solution and then use some method for detecting these annealed sequences. The most predominant means of detection are to use nano- or micro-particles. The particles can be encoded in a number of ways to allow for indexing. Any method that can be used to specifically encode particles could be used, but most employ optical/spectral codes, graphical/patterned codes, shapes or compositions. The particles can be directly linked to probes or used in a secondary step for detection. This secondary step can also follow a solution-based sequence specific enzymatic reaction to determine the target genotype followed by capture onto the solid microsphere surface for detection. Reactions that may be used are allele-specific primer extension (ASPE), oligonucleotide ligation assay (OLA) and single base chain extension (SBCE). Commercial kits to employ any of these approaches are available through Luminex, Inc using their spectrally encoded bead system (Duncan, et al. (2008) 67th Annual Meeting of the Society-for-Developmental-Biology 312, incorporated herein by reference). The protocols for such assays are well known to those skilled in the art and could be developed or modified to identify and quantitate the presence of numerous sequences.

In other embodiments, probes are labeled directly or indirectly to facilitate detection following hybridization in solution. The nucleic acids may be labeled in any way that facilitates detection including optical, sequence or mass-related properties. The Nanostring technology relies on unique single stranded DNA tag regions that have been hybridized to RNA probes labeled with specific fluorophores to provide spectral bar coding that can be detected at the single molecule level using optical microscopy (Geiss, et al. (2008) Nat Biotechnol 26: 317-25, incorporated herein by reference). DNA barcodes attached to probes also allow solution-based hybridization, but read-out is through sequencing or chip arrays. MassCode technology uses probes that have distinct molecular weight tags that can be released by UV exposure (Richmond, et al. (2011) Plos One 6: e18967, incorporated by reference). A variety of labeling and detection methods may be used to identify probes that have annealed to target sequences for the application in this disclosure.

In cases in which a hybridization-based method is used, the number of targets that are assayed can vary from only one target sequence from each chromosome will be will included to identify whole chromosomal aneuploidies (i.e., 24 target sequences) to more than thousands. More target sequences will enhance the sensitivity, specificity and resolution of these assays. The number of target sequences may be more than 24, 50, 100, 200, 500, 1000, 5000, 10,000, 50,0000, 100,000, 500,0000 or 1,000,000.

### V.G. Sample Preparation and Raw Data Generation for Amplification-Based Transcriptome Profiling

In other embodiments, methods for identifying and quantitating transcript levels are performed using an amplification-based method. In many cases, the amplification method will be PCR, but a variety of alternative methods of amplification could used in place of PCR. The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from a number of commercial vendors. There are 2 general amplification-based approaches to determine the amount of template in a sample, quantitative amplification and digital amplification.

V.G.i. Quantitative Amplification

Quantitative amplification determines the amount of template based on the number of cycles of amplification that are required to cross the threshold of detection. In most cases, this type of quantitation will be performed using PCR as the method of amplification. A guideline of steps for experimental design and data analysis for quantitative PCR (qPCR) analyses are outlined by Bustin, et al. (2009) Clinical Chemistry 55: 611-622, incorporated herein by reference. In most cases, qPCR requires a means to follow the amount of amplification product in real time. This is most commonly achieved through the use of fluorescence based technologies including, but not limited to: (i) probe sequences that fluoresce upon nuclease-catalyzed hydrolysis (TaqMan; Applied Biosystems, Foster City, Calif., USA) or hybridization (LightCycler; Roche, Indianapolis, Ind., USA); (ii) fluorescent hairpins; or (iii) intercalating dyes (SYBR Green).

Fluorogenic nuclease assays are one specific example of a real-time quantification method that can be used successfully in the methods described herein. This method of monitoring the formation of amplification product involves the continuous measurement of PCR product accumulation using a dual-labeled fluorogenic oligonucleotide probe—an approach frequently referred to in the literature as the “TaqMan° method.” (see U.S. Pat. No. 5,723,591; Heid et al., 1996, Heid, et al. (1996) Genome Research 6: 986-994, incorporated herein by reference). It will be appreciated that while “TaqMan® probes” are the most widely used for qPCR, this disclosure is not limited to use of these probes; any suitable probe can be used. Other detection/quantification methods that can be employed in this disclosure include, but are not limited to, (1) FRET and template extension reactions (U.S. Pat. No. 5,945,283 and PCT Publication WO 97/22719), (2) molecular beacon detection (Piatek et al., 1998, Nat. Biotechnol. 16:359-63; Tyagi, and Kramer, 1996, Nat. Biotechnology 14:303-308; and Tyagi, et al., 1998, Nat. Biotechnol. 16:49-53), (3) Scorpion detection (Thelwell et al. 2000, Nucleic Acids Research, 28:3752-3761 and Solinas et al., 2001, Nucleic Acids Research 29:20), (4) Invader detection (Neri, B. P., et al., 2000, Advances in Nucleic Acid and Protein Analysis 3826: 1 17-125 and U.S. Pat. No. 6,706,471) and (5) padlock probe detection (Landegren et al., 2003, Comparative and Functional Genomics 4:525-30; Nilsson et al., 2006, Trends Biotechnol. 24:83-8; Nilsson et al., 1994, Science 265:2085-8), each reference hereby incorporated in its entirety.

In particular embodiments, fluorophores that can be used as detectable labels for probes include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, 6-Fam™ are all available from Applied Biosystems, Foster City, Calif.).

Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, incorporated herein by reference.

In particular embodiments, combined thermal cycling and fluorescence detecting devices can be used for precise quantification of target nucleic acids. In some embodiments, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product.

According to some embodiments, one can simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target nucleic acid. By acquiring fluorescence over different temperatures, it is possible to follow the extent of hybridization. Moreover, the temperature-dependence of PCR product hybridization can be used for the identification and/or quantification of PCR products. Accordingly, the methods described herein encompass the use of melting curve analysis in detecting and/or quantifying amplicons. Melting curve analysis is well known and is described, for example, in U.S. Pat. Nos. 6,174,670; 6,472,156; and 6,569,627, each of which is hereby incorporated by reference. In illustrative embodiments, melting curve analysis is carried out using a double-stranded DNA dye, such as SYBR Green, Eva Green, Pico Green (Molecular Probes, Inc., Eugene, Oreg.), ethidium bromide, and the like (see Zhu et al., 1994, Anal. Chem. 66: 1941-48, incorporated herein by reference).

Those skilled in the art will appreciate that specific primers will need to be designed to facilitate quantitative evaluation of sequences derived from target transcripts. In most cases, these primers will have been validated empirically to determine amplification efficiency prior to use. In some cases, these primers will be chosen from databases or commercially available catalogs, in other cases, the primers will be custom synthesized. The number of target sequences to assays will depend upon the resolution that is desired. In some cases, only one target sequence from each chromosome will be will included to identify whole chromosomal aneuploidies (i.e., 24 target sequences). In other cases, many more than 24 target sequences will be included to enhance the sensitivity, specificity and resolution of these assays. The number of target sequences may be more than 24, 50, 100, 200, 500, 1000, 5000, 10,000, 50,0000, 100,000, 500,0000 or 1,000,000.

According to certain embodiments, one can employ an internal control to quantify the amplification product indicated by the fluorescent signal. See, e.g., U.S. Pat. No. 5,736,333, incorporated herein by reference.

In certain embodiments, a preamplification step is performed prior to the qPCR to enhance the number of target sequences that may be assayed and/or to introduce tags on specific nucleic acids. Typically, preamplification prior to qPCR is performed for a limited number of thermal cycles (e.g., 5 cycles, or 10 cycles) to provide quantitative amplification of the nucleic acids in the reaction mixture. In certain embodiments, the number of thermal cycles during preamplification can be 3, 4, 5, 6, 7, 8, 9, 10, 11, 12, 13, 14, 15, or more than 15. In other cases, alternative means of quantitative amplification are used. In some cases, a preamplification step is not performed.

V.G.ii. Digital Amplification

In digital amplification, a limiting dilution of the sample is made across a large number of separate amplification reactions such that most of the reactions have no template molecules and give a negative amplification result. In counting the number of positive amplification results, e.g., at the reaction endpoint, one is counting the individual template molecules present in the original sample one-by-one. A major advantage of digital amplification is that the quantitation is independent of variations in the amplification efficiency—successful amplifications are counted as one molecule, independent of the actual amount of product. In some cases, the amplification method will be PCR. For discussions of “digital PCR” see, for example, Vogelstein and Kinzler (1999) Proceedings of the National Academy of Sciences of the United States of America 96: 9236-9241; McBride et al., U.S Patent Application Publication No. 20050252773, incorporated herein by reference.

In certain embodiments, a preamplification step as described above for quantitative amplification is performed before digital quantitation. In some embodiments, there will not be a preamplification step prior to digital amplification.

For digital amplification, aliquots of the sample will be distributed to separate amplification reactions such that each individual amplification reaction is expected to include one or fewer amplifiable nucleic acids. One of skill in the art can determine the concentration of targets in the sample and calculate an appropriate amount for use in digital amplification. More conveniently, a set of serial dilutions of the targets can be tested. In some cases, identical (or substantially similar) amplification reaction conditions are run for all of the assays. In other cases, a variety of amplification conditions optimized for each individual reaction are performed. Any amplification method may be employed, but conveniently, PCR may be used, e.g., real-time PCR or endpoint PCR. Amplification products may be detected, for example, using a universal probe, such as SYBR Green, or target- and reference-specific probes, which may be included in all digital amplification mixtures. In some cases, only one target sequence from each chromosome will be assayed to identify whole chromosomal aneuploidies (i.e., 24 target sequences). In other cases, many more than 24 target sequences will be included to enhance the sensitivity, specificity and resolution of these assays. The number of target sequences may be more than 24, 50, 100, 200, 500, 1000, 5000, 10,000, 50,0000, 100,000, 500,0000 or 1,000,000.

A variety of approaches and devices may be used to perform these multiplexed reactions. Digital amplification methods can make use of certain-high-throughput devices suitable for digital PCR, such as microfluidic devices typically containing a large number of small-volume reaction sites (e.g., nano-volume reactions). These reaction mixtures may be performed in a reaction/assay platform or microfluidic device or can exist as separate droplets, e.g., as in emulsion PCR. Illustrative Digital Array™ microfluidic devices are described in U.S. applications owned by Fluidigm, Inc., such as U.S. application Ser. No. 12/170,414, incorporated herein by reference. Methods for creating droplets having reaction component(s) and/or conducting reactions therein are described in U.S. Pat. No. 7,294,503, U.S. Patent Publication No. 20100022414, U.S. Patent Publication No. 20100092973, incorporated herein by reference. Any technology that allows for high throughput means to set up, perform and monitor amplification reactions may be used.

### VIII. Generating Regional Expression Count Data for Loci and Alleles from Raw Data

Following generation of the raw transcriptome data, the raw sequencing data are then processed to generate regional expression counts. Regional expression counts provide a quantitative assessment of the amount of RNA produced from pre-determined regions of a reference sequence in a sample. In some cases, the pre-determined regions may be the defined by biologic boundaries such as loci, isoforms of loci, alleles or exons. In other cases, the pre-defined region may be specified lengths of nucleotides within a locus. Since the amount of input RNA may vary from samples, another essential process in generating regional expression count data is to normalize the data.

VIII. A. Generating Regional Expression Count Data for Loci and Alleles from RNA-Seq Data

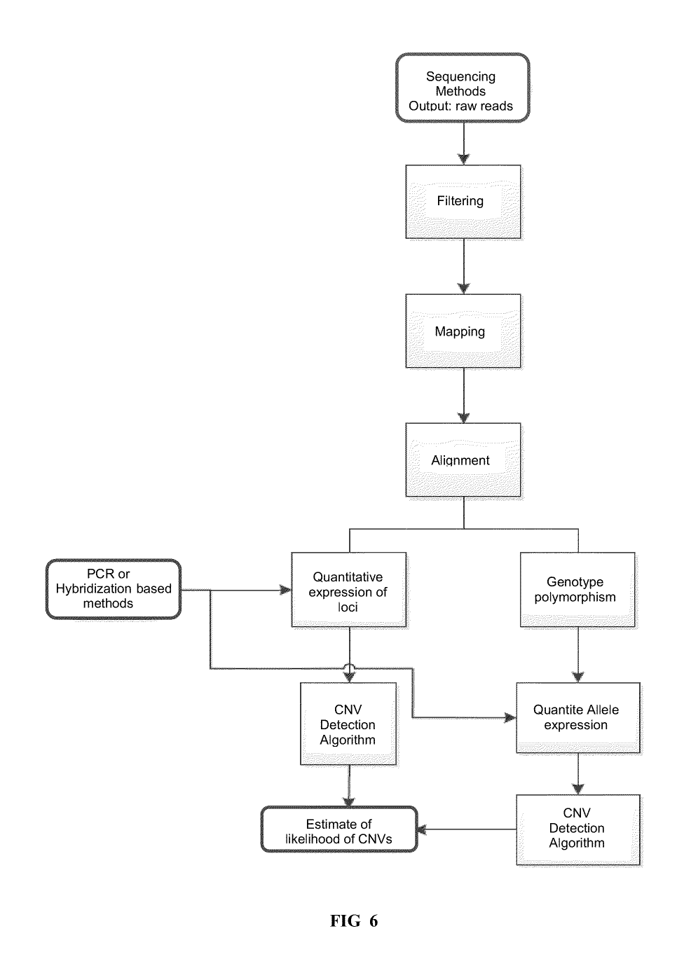

Since RNA-Seq generates raw sequence data, several steps must be followed to convert these data into regional expression counts that include quality assessment, data filtering, sequence alignment and generation of expression count data for locus and alleles (FIG. 6).

VIII.A.i. Quality Assessment and Data Filtering

After sequencing, reads may be assigned a quality score. For example, raw data may be assessed for quality using various informatics tools, including but not limited to available programs such as FastQC version 0.10.0 (http://www.bioinformatics.babraham.ac.uk/projects/fastqc/). In this case, an algorithm may be used to assess quality per sequence and per base (phred scores); GC and N content; sequence length distribution, overrepresented sequences, sequence duplication levels and kmer content. Based on these quality scores, poor sequences and/or segments of sequence are culled. In another example, quality assessment of raw sequence data may be performed by the program SolexaQA. Sequencing reads with a quality score at least 90%, 95%, 99%, 99.9%, 99.99% or 99.999% may be filtered out of the data set. In other cases, sequencing reads assigned a quality scored less than 90%, 95%, 99%, 99.9%, 99.99% or 99.999% may be filtered out of the data set.

VIII.A.ii. Aligning Sequence Reads

Sequence reads that meet a specified quality score threshold are aligned to a reference genome or transcriptome reference sequence to generate aligned sequence reads. In some cases, a reference genome may be a genomic sequence such as genome assemblies from Ensembl or NCBI. In other cases, the sequence reads will be aligned to a transcriptome assembly such as those developed by Ensembl or NCBI. Any program that can accurately and efficiently align RNA-Seq data to the reference sequence may be used. In some programs, indexing of the reference or sample sequence is performed to reduce the computational demands of such searches. In the case of alignments of RNA-Seq data to a genome reference sequence, it is also necessary for mapping algorithms to be able to identify introns. Examples of programs that may be used include TopHat, SplitSeek, SOAPals, SpliceMap, SplitSeek, QPALMA/GenomeMapper/PALMapper, Passion, RNA-Mate, RUM, SOAP Splice, Supersplat and HMMSplice (Garber, et al. (2011) Nat Methods 8: 469-477, incorporated herein by reference).

Alternatively, the transcripts may be mapped to a transcriptome database such as Ensembl. For this type of mapping, any aligner that has been developed for mapping DNA reads to the genome (i.e., not designed for reads with splice events) may be used. This technique may include the use of additionally alignment software such as MAQ, BWA, PASS, SHRiMP, RMAP, SOAP2, ELAND, SeqMap, ZOOM, MOM, Vmatch, Cloudburst, AB map reads, MuMRescueLite, Novoalign, Zoom, Mosaik (Horner, et al. (2010) Briefings in Bioinformatics 11: 181-197 and Fonseca, et al. (2012) Bioinformatics 28: 3169-77).

After sequence reads have been aligned, assembly programs may be used to generate a transcriptome assembly. Such programs assemble the alignments into a parsimonious set of transcripts and can predict novel genes and isoforms according to the read mapping results on the reference genome. Examples of assembly programs are Cufflinks, G-MO.R-Se, Cufflinks, Scripture, ERANGE Multiple-K, Rnnotator, Trans-ABySS, Oases and Trinity (Martin and Wang (2011) Nat Rev Genet 12: 671-682, incorporated by reference).

VIII.A.iii. Generation of Regional Expression Counts for Loci

Once aligned sequence reads have been generated, it is necessary to enumerate the number of sequence reads within pre-determined regions of the reference sequence, thereby generating an expression count. In some cases, these pre-determined regions may be the defined by biologic boundaries such as loci, isoforms of loci or exons. In other cases, these predetermined windows may be specified lengths of nucleotides within each locus. In some cases, combinations of more than one type of pre-determined regions may be used.

In some cases, the commonly used Cufflinks program is used to determine read counts for loci. Cufflinks and an additional program, Cuffdiff, implement a linear statistical model to estimate an assignment of abundance to each transcript. This estimate explains the observed reads with maximum likelihood. Cufflinks and Cuffdiff calculate the expression level of each alternative splice transcript of a gene and sums the expression level of each splice variant. This estimate of gene expression is directly proportional to other techniques for measuring gene expression such as RPKM or FPKM. A number of other quantitation tools may be used for quantitating gene expression, such as rpkmforgenes and BEDTools.

In other cases, read coverage data will be determined per base, allowing for determinations of read counts in other user-specified predetermined windows. To generate depth of coverage information of each base, PILEUP files can be generated using SAMtools.

VIII.A.iv. Generation of Regional Expression Counts for Alleles of Polymorphisms

In some cases of the composition and methods of this disclosure, it may be useful to generate expression counts, for alleles rather than loci. To assess the expression of alleles it is necessary to evaluate the expression of polymorphisms. In most cases, the polymorphisms that are used are single nucleotide polymorphisms (SNPs), which are present in coding regions at a frequency of about 1 every 300 basepairs. To genotype coding SNPs in a sample, the focus is on identifying heterozygous SNPs as these are the ones that would not be identified with standard mapping algorithms where there is some leeway for mismatches. As a first step to identifying heterozygous SNPs, the depth of coverage for each base is determined. This parameter provides a confidence score for calls and may be generated by any suitable algorithm, such as SAMTools software. Variant sites may then called by any algorithm that can identify and call variants. One such example is Genome Analysis Toolkit software. Alternative software for SNP genotyping that may be used include, but are not limited to, SOAPsnp, MAQ, samtools and Beagle.

In other embodiments, other polymorphic variations such as indels (small insertions or deletions) may be identified in addition to SNPs to distinguish alleles. Generally, any type of polymorphism or combination of types of polymorphisms may be used to generate allelic information.

Once alleles have been distinguished by polymorphisms, the relative expression of each allele can be determined using any algorithm that can determine expression levels from these data such as the approaches described herein for determining locus expression levels. Since polymorphisms have defined locations within the genome, the pre-determined window for expression counts for these alleles will simply be the bases involved in the polymorphism. For example, in SNPs, the window will only be one base pair.

VIII.B. Generating Regional Expression Data from Hybridization-Based Methods for Loci and Alleles of Polymorphisms

Since hybridization-based methods use probes with defined genome coordinates, the analysis for hybridization array-based data requires fewer steps. Hybridization-based methods are prone to systemic biases related to properties of hybridization, so data must be normalized to remove non-relevant effects such as the GC content of the target sequence, probe specific intensity bias due to difference in binding affinity and spatial artifacts. Normalization may be performed using methods that include, but are not limited to, mean-signal, spike-in or quantile normalization. In cases in which more than one probe is present within the locus, all probe data may be presented or probes from each locus may be compressed to a single locus value using weighted averaging or other appropriate methods. In some cases, these data may then used for subsequent analyses. In other cases, these expression levels may be normalized to the expression levels of one or more loci expressed within the sample. For determining expression counts, signals in predetermined windows are then tabulated using any algorithm capable of doing these calculations. Pre-determined regions that may be used include the locus, isoform, exon or sequence to which the probe anneals. In the case of probes for identifying polymorphisms, the predefined region will be the variant base pair. In some cases, the polymorphisms evaluated will be SNPs, in which the pre-determined region will be 1 basepair. There are a variety of software packages available for hybridization-based detection methods that can genotype SNPs and provide relative intensity data for each allele.

VIII.C. Generating Regional Expression Counts from Amplification-Based Methods for Loci and Alleles of Polymorphisms

Any method that can be used to generate quantitative data reflecting transcript abundance in pre-determined regions of a reference sequence from raw data generated by amplification-based quantitation methods. The pre-determined regions for the evaluation of locus expression include, but are not limited to, loci, isoforms, exons or sequence that is amplified. The predetermined region for polymorphisms will be the variant bases.

In some cases of qPCR, quantitation will be absolute, based on the use of a standard curve generated by determining threshold cycles for a range of defined concentrations of one or more control RNA. In other cases, quantitation will be relative, with results being expressed as a ratio to an external reference sample known as a calibrator. Methods for relative quantitation include, but are not limited to, the standard curve, comparative Cₜ(2^{-ΔΔCt}), Q-gene, Gentle et al, Pfaffl, Liu and Saint, and DART-PCR models as described by Wong and Medrano (2005) Biotechniques 39: 75-85. Since different samples will likely differ in the amount of input RNA, it necessary to normalize to one or more transcripts from the sample that serve as internal controls. Internal control may be chosen from standard lists of such controls or identified empirically using methods such as those described by Bustin, et al. (2005) Journal of Molecular Endocrinology 34: 597-601 and Wong and Medrano (2005) Biotechniques 39: 75-85.

For digital PCR, absolute numbers of target sequence will be determined through the use of a one or more standard curves generated using control samples with defined numbers of copies of target sequence.

### IX. Identification of CNVs

Following generation of regional expression count (REC) data from loci and alleles of polymorphisms from RNA-Seq, hybridization- or PCR-based methods, the data are analyzed in a two-step process to identify CNVs. Generally, the first step is to compare the REC data generated from the embryo to a reference. This step determines whether each region that defines a REC has higher, lower or similar expression relative to the reference. From these comparisons, the difference between the sample and reference for each REC is assigned a value reflecting the the difference between the embryo and the reference, known as a relative regional expression values (RREV). For example, fold change may be used. If the REC for one region in the embryo is 25 and the corresponding REC is 10 in the reference, then the RREV would be 2.5. The RREV data are then analyzed to identify regions in which the sample differs from the reference. For example, a region that has a significant bias toward upregulated expression would suggest a gain of copy and region that is down-regulated would suggest a loss of copy relative to the reference.

### IX.A. Locus-Based CNV Identification

For locus based, CNV identification, the locus-based REC data generated from sequencing-, hybridization- or amplification-based methods of transcriptome analysis will serve as input data. In some cases, all REC data that are available for all loci are analyzed. In other cases, only a subset of locus REC data are evaluated. The loci selected for analysis may be due to empirically determined biologic characteristics such as high expression, high correlation with copy number or low biologic variability, which would have beneficial effects on the subsequent analyses.

In the first phase of analysis, the REC data for the embryo will be compared to corresponding REC data from the reference. For the purposes of comparing the REC values from a sample to those of a reference, any reference that can facilitate inference of copy number in the test sample may be used. In some cases, the reference may be REC data from a single embryo. In other cases, the reference may be derived from REC data from more than 1, 5, 10, 50, 100, 1000, 5000, 10,000 embryos. In some cases, the reference may be derived from one or more embryos in which genotypic information is available pertaining to the genome copy number status for some or all of the loci that are evaluated. In other cases, the reference may be generated from one or more embryos in which there is no genotypic information available. In some cases, the embryos comprising the reference may be matched to the sample based on biologic factors that might affect embryonic gene expression. Such factors include, but are not limited to (1) biologic conditions of one or both parents such as age, health status, genotype, diet, body habitus, history of illness or environmental exposure, (2) the specific assisted reproductive methods used to produce the embryo(s) such as ovarian stimulation protocol, method of gamete retrieval, technique of fertilization, embryo culture conditions and biopsy method and (3) the methods used to generate the transcriptome data. In some cases in which more than one embryo is used for generating the reference REC values, the reference REC values will represent the median value of the RECs in the reference set. In other cases, the reference REC will be derived from the means of values in the dataset.

As a result of the comparison of the embryo REC data to that of the reference, data will be output that reflects the relative differences between the embryo and the reference, data referred to as relative regional expression values (RREVs). Any value that qualitatively or quantitatively captures this comparison may be used. In some cases, the RREVs may be the absolute differences from the reference (i.e., sample REC−reference REC). In some cases, these RREVs will be used directly for subsequent analyses. In other cases, only absolute differences beyond certain thresholds will be presented. The threshold for upregulation may be greater than a 1, 5, 10, 20, 25, 30, 35, 40, 50, 75, 100% change. The threshold for down-regulation may be a 1, 5, 10, 15, 20, 25, 30, 35, 40, 45, 50, 55, 60, 65, 70, 75, 80, 85 or 90% change. Expression levels inside of the two threshold boundaries would be considered similar to the reference. The threshold may be set arbitrarily or based on empiric data or modeling.

In other cases, the RREVs may be fold-changes (i.e., embryo REC divided by REC or reference REC divided by embryo REC). In some cases, the fold-change data will be used directly for subsequent analyses. In other cases, threshold(s) will be applied to assign up- or down-regulation or no change. The threshold for upregulation may be a ratio greater than 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5 or 3. Threshold for down regulation may be less than 1, 0.95, 0.9, 0.85, 0.8, 0.75, 0.7, 0.65, 0.6, 0.55, 0.5, 0.45, 0.4, 0.35, 0.3, 0.25, 0.2, 0.15 or 0.1. Expression levels not outside of the upper and lower threshold values will be considered as no-change. In some cases, the thresholds are determined by the user. In other cases, the thresholds are based on reference data.

In other instances, a sign may be applied to difference between the embryo and the reference. For example, RREVs based on absolute differences or ratios may be assigned a qualitative value of + for values above a threshold, − for values below a threshold and 0 for values in between the threshold. The threshold for upregulation may be set a value that may be greater than 1, 2, 5, 10, 15, 20, 25, 30, 35, 40, 50, 60, 70, 80, 90, 100, 125, 150, 175, 200, 225, 250, 275, or 300% of the reference value. The threshold for down-regulation may be set to be lower than 1, 2, 5, 10, 15, 20, 25, 30, 35, 40, 50, 60, 70, 80 or 90% of the reference value.

In some cases, thresholds for RREVs may be set based on standard deviations of the reference data. The upper threshold may be set at more than 0.1, 0.2, 0.3, 0.4, 0.5, 0.6, 0.7, 0.8, 0.9, 1, 1.5, 2, 2.5, 3, 3.5, 4, 4.5 or 5 standard deviations above the reference mean. The lower threshold may be set at below 0.1, 0.2, 0.3, 0.4, 0.5, 0.6, 0.7, 0.8, 0.9, 1, 1.5, 2, 2.5, 3, 3.5, 4, 4.5 or 5 standard deviations below the reference mean.

Once RREVs have been generated from the comparison of the embryo REC data to the reference REC data, an algorithm is applied to look for regions in which there is a regional abnormality of the RREVs. For example, a finding that the fold-change RREVs are around 1.5 for chromosome 21 while the rest of the genome is around 1.0 would indicate that the embryo has trisomy 21. Any algorithm capable of identifying regional biases in RREVs may be used. In some cases, the RREV data may be preprocessed to improve the quality of data prior to subsequent analysis. In some cases, the data may be transformed using base level log ratios to correct for GC content (Li, et al. (2012) Bioinformatics 28: 1307-1313, incorporated herein by reference). In other cases, principal component analysis may be used to remove variance (Fromer, et al. (2012) Am J Hum Genet 91: 597-607, incorporated herein by reference). In other cases, single value decomposition may be used to remove influencing single values (Krumm, et al. (2012) Genome Res 22: 1525-32, incorporated herein by reference).

A diverse array of approaches has been used to identify regional abnormalities in RREVs. In general, there are 2 approaches that may be used: (1) analyzing RREVs over predefined genomic segments that cover the entire genome or (2) using algorithms that evaluate all RREV data and identify regions iteratively to identify region with similar values. In some cases, the predefined segments are static (e.g., whole chromosomes or chromosomal arms). In other cases, there is a predefined sliding window that is then moved by defined distances so that the entire genome is covered. In other cases, combinations of these methods of evaluating the expression profiles of the genome may be used.

In cases in which predefined genomic segments are used, a statistical method is then applied to each segment to determine if there is a statistically significant alteration in the RREVs in that segment as compared to the reference data set. Examples of such methods that can be used depending upon the form of the RREV data include, the sign Z test (Crawley and Furge (2002) Genome Biology 3: 1-8), Fisher exact test (Hosack, et al. (2003) Genome Biol 4: R70; Kano, et al. (2003) Physiological Genomics 13: 31-46), mean and variance permutation tests, t test (Yi, et al. (2005) Genomics 85: 401-412) and hidden Markov modeling (Amarasinghe, et al. (2013) BMC bioinformatics 14 Suppl 2: S2; Fromer, et al. (2012) Am J Hum Genet 91: 597-607; Geng, et al. (2008) Bioinformatics Research and Applications 4983: 414-425; Love, et al. (2011) Statistical applications in genetics and molecular biology 10; Plagnol, et al. (2012) Bioinformatics 28: 2747-2754), citations herein incorporated by reference. In some cases, the regions with similar patterns of locus expression are then combined using circular binary segmentation (Deng and Disteche (2010) Plos Biology 8; Koboldt, et al. (2012) Genome Res 22: 568-76; Sathirapongsasuti, et al. (2011) Bioinformatics 27: 2648-2654, incorporated herein by reference).

In other cases, the entire RREV data set is evaluated systematically to identify regions with similar expression patterns. In some cases, a clustering method is used to identify and then build regions with similar expression patterns (Sharan, et al. (2003) Bioinformatics 19: 1787-1799, incorporated herein by reference). In other cases, expectation maximization algorithms are used to define the boundaries of regions (Myers, et al. (2004) Bioinformatics 20: 3533-43, incorporated herein by reference). In other cases, a piecewise constant fit algorithm is used to define regions (Lingjærde, et al. (2005) Bioinformatics 21: 821-822, incorporated herein by reference).

### IX.B. Allele-Based CNV Identification

In some cases, CNVs may be identified by analyzing the expression of alleles from transcribed loci. In most instances when CNVs are present, only one allele of a locus is altered. This copy number change may impact the expression of the affected allele, and for loci that have 2 distinguishable alleles, the ratio of expression between the alleles will be altered. In deletions, an allele will be lost. For hemizygous loci (i.e. monoallelic loci), the polymorphisms associated with the locus will be absent. For loci that are normally biallelic, there will be only a single allele. Consequently, autosomal regions with deletions will have monoallelic expression, also known as loss of heterozygosity (LOH). LOH may also arise if there is a type of uniparental disomy (UPD) in which there are two copies of the same chromosomal homologue.

A gain in copy number for a monoallelic allele will increase its copy number by 2-fold and for biallelic loci, a gain will alter the ratio for heterozygous loci from the 1:1 to 2:1 or 1:2 and will result in a 50% increase in copy number for homozygous loci.

These alterations in copy number of alleles will also be reflected in the expression of the alleles. Deletions may be detected by identifying genomic regions on hemizygous chromosomes (i.e., most of the X and Y chromosomes in mammalian males) that lack detectable polymorphisms in the affected region. Whereas deletions in autosomal chromosomes will cause LOH. LOH due to deletions may be distinguished from those associated with UPD based on the level of expression of the allele: deletions should have half of the level of expression of the loci whereas UPD will have normal levels of expression from loci. Copy number gains of a genomic region may be identified through an increase in expression of alleles on the strand of DNA that has increased in copy number. For gains that increase the copy number of one of the two alleles of heterozygous loci, gains may also be detected by alterations in the ratio of expression of the two alleles from 1:1 to 2:1 or 1:2.

The approaches to using allelic information can be divided into approaches that can be used in situations in which the haplotypes of the embryo have or have not been determined. When the haplotypes are determined, the alleles which are co-localized together on a particular chromosome are identified. Haplotyping of embryos based on transcriptome data can be achieved through identification of polymorphisms in the embryo transcriptome data combined with genotypic data from family members of the respective embryos and/or computational approaches based on haplotype data from populations or unrelated individuals (see Browning Browning and Browning (2011) Nature Reviews Genetics 12: 703-714 for a review of haplotyping methods, incorporated herein by reference). In some cases, the haplotypes may be phased, in which the haplotypes are linked to the parent of origin (i.e., maternal or paternal).

In an embryo in which the haplotypes have been determined, it may be possible to look for evidence of alterations in expression of alleles on the same chromosome. For the autosomes, this would be performed by evaluating the expression of all transcribed polymorphisms that are associated with each chromosomal haplotype separately.

Algorithms similar to those described previously for evaluating locus expression could be used to identify regional disturbances in expression of alleles for each of the two haplotypes. Regional expression counts for alleles from the same chromosomal haplotype would be compared to corresponding reference RECs from haplotype regions. With the exception of requiring haplotype determined samples for the reference, all of the possible types of references described herein for locus-based REC data generation may be used. Once RREV data for haplotypes for each chromosome in the embryo and then assembled, it would be possible to analyze these data using the same algorithms described herein for detection of CNVs using locus RREV data.

In other cases in which embryos are haplotyped, allelic expression of the autosomes may be assessed by evaluating the relative expression of the alleles for each locus from one haplotype relative to the other. The relative expression of the two alleles is presented as an allele expression ratio (AER). Ratios are generated by dividing the expression level of allele from one haplotype by that from the other haplotype. To identify alterations in these ratios, the ratios from the test sample would then be compared to a reference dataset. The reference may be the expected AER (e.g., 1 for autosomal regions and the X chromosome in female embryos and a ratio of 0 or 1 for the X and Y chromosomes in males) or may be based on empiric AER data from a reference set of haplotyped embryos as described above for analyses of allelic expression may be used. A variety of statistical analyses can be used to determining if allelic ratios of the sample differ significantly from those of the reference(s). In some cases, ratios will be transformed or processed prior to the comparison to reduce noise, account for biases introduced by the technique, correct for mosaicism or eliminate any other influences that do not pertain to allelic expression. In other cases, the AERs will not be transformed. In some cases, a binomial test will be performed to determine if the sample AER differs significantly from the reference AER. In some cases, the results will be corrected for multiple testing using FDR or similar correction. In some cases, error parameters for miscalling genotypes will be included as described by Nothnagel, et al. (2011) Human Mutation 32: 98-106, incorporated herein by reference. In other cases, a Bayesian model developed by Skelly et al (Skelly, et al. (2011) Genome Res 21: 1728-1737, incorporated herein by reference) may be used in place of the binomial test to identify allelic imbalance. In cases in which statistical analyses are performed, AERs from the embryo may be considered to differ from the reference AER if the p value is less than 0.1, 0.05, 0.01, 1E-2, 1E-3, 1E-4, 1E-5, 1E-6, 1E-7, 1E-8 or 1E-9. In some cases, a difference of more than 1, 2, 5, 10, 15, 20, 25, 30, 35, 40, 50, 60, 70, 80, 90, 100, 125, 150, 175, 200, 225, 250, 275, or 300% may be considered to indicate that the embryo AER differs from the reference AER. In some cases, statistical analyses are performed on more than one AER to improve accuracy due to the noise of the system.

Following individual analyses of AERs, some or all of the data may be combined to identify contiguous regions that differ significantly between the embryo and the reference. In one approach, a defined window of a certain number of SNPs may be chosen to identify allelic bias. In other cases, groups of AERs may be analyzed by approaches such as (1) simple smoothing: the log of the AER for a SNP is determined by averaging the log AER for the SNP and a defined number of neighboring SNPs, (2) Z-score approach: assigning Z scores for the AERs for each SNP and then determining Z scores of windows of consecutive SNPs, (3) ergodic hidden Markov model (HMM): models genomic state based on HMM states of total expression and allelic ratios of the sample and (4) left-to-right HMM: models genomic state based on models from expression and AERs from all samples. These HMMs also can take into account that AERs would be expected to be consistent across a transcript (Wagner, et al. (2010) Plos Computational Biology 6: e1000849, incorporated herein by reference).

In other cases, allelic expression data will be analyzed without the benefit of haplotype information. In this scenario, allelic expression ratios (AER) can be used to identify abnormalities of allelic expression. In some cases, the AER may be the A/B ratio, determined by dividing the expression of the reference ‘A’ allele by the nonreference ‘B’ allele. In other cases, B allele frequency (BAF; B allele expression level/A+B allele expression levels) will be used. Any ratio that reflects the relative expression of the 2 alleles may be used. Since it is not known which alleles are co-localized to a chromosome, it is necessary to identify regions in which the AERs are skewed significantly from the reference. The reference may be any of those described above for evaluating AER in haplotyped embryos.

In many of the above methods for identifying CNVs, a p value is supplied for each CNV. These values may be supplied with the results to express the probability of the finding. In some cases these p values will be corrected for multiple testing. In other cases, a CNV may be reported as simply being present or not based on a cut-off for p values, corrected or uncorrected, such that p values above 1E-9, 1E-8, 1E-6, 1E-5, 1E-4, 1E-3, 1E-2 or 1E-1, are not considered present. In other cases, user defined criteria for selecting CNVs may be used. In other cases, other clinical data such as data embryo development, morphology and metabolism may be incorporated to modify the probability of the finding be a false positive or negative result. In other cases, the positive and negative predictive values of these analyses may be derived from clinical studies in which confirmatory genome analyses are performed in conjunction with this test.

These methods for screening CNVs may detect a variety of abnormalities in the early embryo. Any form of aneuploidy should be readily detected. Segmental aneusomies, gains or losses of large segments of chromosomes should also be identified. The lower limits of the size of CNVs that can be detected by these approach will vary, depending on a number of factors that include, but are not limited to, the stage at which the embryo is sampled, the size of the sample, the method used to evaluate the transcriptome, the depth and breadth of the coverage of the analysis of the transcriptome and the analytic algorithms used to detect CNVs. It is also likely that this method may be able to detect alterations in ploidy based on disproportionate transcriptional response of select loci to this condition.

It is well established that there is a high frequency of genetic mosaicism in early embryos. Mosaicism is a condition in which one or more genetic alterations are present in only a subset of cells. The most common mechanism for this finding is the development of the genetic alterations in a cell of the embryo after the first mitotic division. This could also be the case for genetic alterations detected by transcriptome analysis in early embryos. It is conceivable that mosaicism will be detected using this diagnostic approach in cases in which there is a substantial representation of both genotypes in the sample analyzed.

### X. Interpretation of Genetic Alterations

Following the identification of genetic alterations such as CNVs, the relevance of the genomic abnormality may be assessed to determine if it is likely pathogenic or benign. To determine the impact, databases that catalog genomic variants such as ENSEMBL (http://www.ensembl.org), the database of chromosomal imbalance and phenotype in humans using ensembl resources (DECIPHER, http://www.sanger.ac.uk/PostGenomics/decipher/), and the database of genomic variants (DGV http://projects.tcag.ca/variation) may be consulted to determine if there may be phenotypic or health effects as a results of the genetic alteration. Other factors that may be considered in assessing the biological impact of a CNV include the size of the CNV, genomic content, evidence of dosage sensitive genes in the online Mendelian inheritance in man (OMIM) database (www.ncbi.nlm.nih.gov/omim) Review of current literature may also provide insight. In some cases, genomic analysis may be performed on the parents to determine if either possesses the observed abnormality. Based on some or all of these analyses, an estimation of the likelihood of the pathogenicity of a CNV may be determined.

Another approach for interpreting the biologic effects of CNVs that may be utilized relates to identifying secondary alterations in transcriptome data (i.e., alterations that are not directly related to the change in copy number such as alterations in the expression of loci from unaffected genomic regions). Transcriptome analyses of whole chromosomal aneuploidies from a variety of species have revealed secondary alterations in the transcriptome that are associated with a generalized cellular stress response. The identification of secondary responses in samples with evidence of CNVs would provide both support for the existence of the CNV and insight into the potential biologic effects of the CNV. Secondary effects may be identified by differential expression algorithms as described herein as well as gene set enrichment analyses such as pathway and ontology analyses as described by Yue and Reisdorf (2005) Curr Mol Med 5: 11-21, incorporated herein by reference. Any methods for identifying secondary changes in the transcriptome could be utilized for this purpose.

### XI. Applications

### XI.A. Detection of Chromosomal Abnormalities

Generally, the compositions and methods of this disclosure may be directed toward detection of CNVs. The most prevalent class of CNVs in early human embryos is aneuploidy, which involves gains or losses of chromosomes. Most of these aneuploidies are lost in the early prenatal period. Approximately half of spontaneous abortions are aneuploid, making this genetic condition the leading known cause of miscarriage. Aneuploidies are present in about 4% of stillbirths and 0.4% of liveborns. Only a very small subset of aneuploidies is compatible with livebirth, mainly consisting of trisomies 13, 21 and 18 and the sex chromosomal abnormalities XO, XXY and XYY.

There are a number of clinical benefits to detecting chromosomal abnormalities in embryos prior to establishing a pregnancy. First, such genetic screening will improve outcomes of assisted reproductive technologies. The detection of aneuploid embryos and the avoidance of transferring these embryos to the uterus will improve the pregnancy rates. Second, this screening is likely to lower the rate of multifetal pregnancies produced by ART. In the US, almost 30% of ART pregnancies are multifetal, mainly due to the fact that more than one embryo is transferred in most ART cycles. Multifetal pregnancies are associated with increased risks of numerous medical complications to the mother, fetus and newborn. Third, screening for chromosomal abnormalities will reduce the risks for having liveborn children with aneuploidy.

### XI.B. Early Detection of Segmental Aneusomies

The compositions and methods of the disclosure may also be used to detect subchromosomal alterations in copy number in embryos. Studies of embryos have also found a high prevalence of copy number alterations that involve large portions of chromosomes, particularly toward the ends of chromosomes. There also is a wide array of smaller genomic imbalances that are relatively common and cause debilitating conditions. Examples of such genomic disorders include: the 3 Mb deletion of 22q11.2 that causes DiGeorge and velocardiofacial syndromes, the 5 Mb deletion of 15q11 that causes Angelman or Prader Willi syndrome depending upon parent of origin, the 1.5 Mb deletion of 17p that causes Charcot-Marie-Tooth syndrome, the 1.5 Mb duplication of 17p that causes hereditary neuropathy and liability to pressure palsies, and the 1.5 Mb deletion of 7q11 that causes Williams syndrome. Given that most of these deletions impact the copy number of more than 20 loci, it is likely that many will be able to be detected through the transcriptome analyses described herein.

### XI.C. Early Detection of Uniparental Disomies

Uniparental disomy (UPD) occurs when there are 2 copies of a chromosome present, and both chromosomal homologues are inherited from the same parent. In cases in which both homologues are identical, it is referred to as isodisomy and in cases in which the chromosomes differ, representing the two different homologues present in one parent, it is referred to as heterodisomy. Uniparental disomy can arise due to errors in the meiotic and early embryonic mitotic divisions. The most common mechanisms are rescues of trisomies and monosomies. In trisomy rescue, a trisomic zygote subsequently loses the single chromosome from one parent, leaving two homologues from the same parent. In monosomy rescue, the sole homologue is duplicated. UPD has effects on any chromosome that is subject to genomic imprinting. Genomic imprinting is defined as the differential expression of genes depending upon from which parent the chromosome was inherited. Only 5 chromosomes have been defined as being imprinted based on clinical phenotypes and basic research: chromosomes 6, 7, 11, 14 and 15. Maternal UPD 6 is associated with transient neonatal diabetes. Maternal UPD 7 is linked to Silver-Russell syndrome. Full UPD for chromosome 11 is presumably lethal, but segmental paternal isodisomic UPD (iUPD) is associated with Beckwith-Wiedemann syndrome. Maternal and paternal UPD 14 are associated with a number of phenotypic and developmental abnormalities. Maternal and Paternal UPD15 represent the most common UPDs. Maternal UPD 15 results in Angelman syndrome and paternal UPD15 causes Prader Willi syndrome. By using methods described herein that can evaluate polymorphisms in the transcriptome, it will be possible to identify UPDs. In the case of iUPD, loss of heterozygosity for the chromosome will be detected in the context of normal expression for the chromosomal loci. For hUPDs, genotypic information from the parents is required to identify that both chromosomal homologues in the embryo were inherited from one parent. The identification of UPD at this early stage would prevent the establishment of pregnancies with this class of disorders, many of which have phenotypic features that impact health and well being.

XI.D. Detection of Other Genetic Alterations in Concert with Large CNVs

Although the methods and compositions described herein are primarily focused on the novel application for genomic CNV detection, the data generated from this type of analysis could also be used in parallel to detect a variety of other types of genetic alterations directly or indirectly. Any alterations that are present in the coding of loci that are expressed in the embryo are amenable to direct mutational detection. These alterations may be associated with disease, disease susceptibilities or traits as mentioned in Section I. A trait is any specific characteristic of an organism that is influenced by its genetics. Examples of traits include genetic diseases (both Mendelian and complex), gender, histocompatibility, susceptibility to disease, height, eye color, intelligence and athletic abilities. One example of how a trait could be identified in the early embryo is the determination of sex of the embryo. The gender of the embryo may be determined through the evaluation of expression of X- and Y-linked loci. For example, an embryo that expresses loci on the Y-chromosome outside of the pseudoautosomal region and expresses X-linked loci at a level consistent with a single copy would indicate male gender. The absence of Y-linked expression and X-linked expression consistent with the presence of 2 X chromosomes (both X chromosomes are active in the preimplantation period) would indicate female gender. Determination of the sex of an embryo is useful in preventing the establishment of pregnancies with X-linked disorders and also for family balancing. Although the focus for this main application is the nuclear genome, transcriptome profiling of cellular total RNA will also allow for assessment of the mitochondrial genome. Genetic alterations that are transcribed from the mitochondrial genome will also be detected. Furthermore, since there are thousands of copies of the mitochondrial genome per cell, analyses of the mitochondrial transcriptome may also be useful in assessing the number of mitochondria per cell.

Although a considerable number of genetic alterations may be directly detected in the transcriptome, there are a substantial portion that will not. Loci that are not expressed or expressed at very low levels will not be able to be identified directly. In these instances, it may be possible to detect these alterations indirectly by one of several methods. In some instances, the inheritance of a genetic alteration such as mutation(s) carried by one or both parents can be determined through linkage analysis. Linkage analysis would allow for the inheritance of genomic regions from the parents to be followed through the inheritance of closely linked polymorphisms. For example, it would be possible to determine if an embryo inherited a mutation that causes Huntington disease from a parent. Huntington disease is an autosomal dominant disorder that is caused by the abnormal expansion of a triplet repeat contained within the HTT (HD) gene. By using informative polymorphisms that are closely linked to this mutation, it will be possible to determine if the mutant or normal allele from the affected parent has been inherited.

A second indirect method for identifying inheritance of a mutation would be to identify an associated haplotype. In this approach, the inheritance of a mutation would be assessed through the determination of whether the embryo contains a haplotype that has been shown to be linked to the mutation. This approach would be most useful for detecting a mutation that recently arose in a small, isolated population. One such example would be the 3398delAAAAG mutation in BRCA2 gene, which has been shown to be linked to one of two rare haplotypes in French Canadians.

The third possible approach to identifying a risk for presence of a genetic alteration would be through the identification of primary or secondary alterations in the transcriptome. It may well be that the mutation, although not transcribed, may impact the expression of one or more loci that are expressed in the embryo.

XI.E. Assessment of Embryo Health and Developmental Potential in Concert with CNV Screening

A very powerful added benefit of using a transcriptome-based method for identifying CNVs is that the transcriptome also provides a tremendous amount of additional information about the health and biological functioning of the embryo. By surveying transcripts associated with various biologic pathways, it may be possible to identify a variety of perturbations that would suggest compromised development, health and/or developmental potential. Abnormalities in the expression of loci that constitute the developmental signature of the stage at which the embryo was biopsied may reveal that the embryo has not developed properly. Examples of such genes in a blastocyst biopsy sample would be the expression of loci involved in specification of the trophectoderm and preparation for implantation as well as imprinted genes that are reprogrammed during this period of development. Abnormalities in other classes of genes that are vital to cellular function, such as those involved in cell division, energy metabolism, biosynthesis, nucleic acid synthesis and repair, stress response, programmed cell death may suggest a compromised state of health. In some cases, the compromised health may be due to genetic abnormalities present in the embryo. In other cases, the compromised health may be due to current or past exposure to adverse environmental factors such as exposure to toxins or other insulting agents, infection or a suboptimal culture environment. In the case of environmental insults, it may be possible to identify the particular insult from the transcriptome data and make changes in the procedures for generating or culturing embryos to avoid or minimize exposure to the insult. In other cases, the compromised health may be due to a combination of genetic and environmental factors. Given the incredible complexities of cellular function and the fact that many features of the transcriptome are not understood, one of the most fruitful approaches may be to identify transcriptome profiles associated with high developmental potential based on data from embryos that developed into healthy offspring. With establishment of these profiles, it may then be possible to use these profiles for identifying embryos with the highest developmental potential. In some cases, these embryos classified as having developmental potential embryos may then be selected for transfer.

### XI.F. Evaluation of Mitochondrial Gene Expression

Since total cellular RNA is the source for these methods, it would also be possible to analyze the mithochondrial transcriptome in the embryonic samples described herein. The mitochondrial genome encodes 13 proteins, 22 transfer RNAs and 2 ribosomal RNAs. In one application, global expression of the mitochondrial transcriptome could be used as a means to evaluate the number of copies present in embryonic cells. The number of mitochondria in human oocytes varies over a more than an order of magnitude, and there is evidence to suggest that there are lower numbers of mitochondria in oocytes that fail to fertilize and in embryos that fail to develop. Quantitation of mitochondrial cellular content may be an important biomarker of developmental competence. It is also known that preimplantation mammalian embryos become more metabolically active during the course of the preimplantation period, and there are data suggesting that there is a range of metabolic activity that correlates with good developmental outcomes. Thus, expression of the proteins involved in energy metabolism may also serve as a marker of health and developmental potential. A number of mutations in the mitochondrial genome that are known to cause human disease are present in coding regions, making them amenable to detection directly in the transcriptome.

XI.G. Combination of Transcriptome Profiling with Other Diagnostic Approaches

A potentially synergistic approach may be to combine transcriptome analysis with other diagnostic approaches that are available or being developed for the preimplantation embryo. One additional analysis would be to include genomic analysis. If one large or two biopsies are obtained, it would be possible to perform analyses on both the transcriptome and genome simultaneously. Performance of both analyses would allow findings of genetic alterations in transcriptome analyses to be confirmed. Genome analysis would also supplement transcriptome analysis by providing higher resolution and more comprehensive analysis of the genome, thereby expanding the spectrum of genetic alterations that could be directly detected. Alternatively, the additional biopsy sample could be used for proteomic analysis to evaluate the profile of proteins that are expressed in the embryo. It is also possible to combine transcriptome analysis with any other methods that are currently being used or developed to assess embryonic health and competence and that do not interfere with transcriptome analysis. Several of the most promising emerging diagnostic approaches are evaluating the developmental progression of the embryo through time lapse imaging and assessing metabolism and secreted protein profiles through analysis of the embryo's culture medium.

### XII. Storage and Dissemination of Embryo Genotypic Information

Transcriptome-based screening of embryos has the capability of generating millions of bits of information pertaining to the health and genetics of an embryo. Furthermore, some information from this analysis may indirectly provide genetic information pertaining to the individual(s) from which the embryo was generated. The massive amount of raw and processed data generated from this analysis may be stored in any manner that allows for archiving and retrieval, most often through memory storage devices accessed by computer. Given that this genetic screening method may be applied to embryos from a number of species including human embryos, there are a wide range of rules and regulations that may govern the use and storage of these data. For clinical testing of human embryos, appropriate consents must be obtained from parties involved in producing the embryo and standard HIPAA regulations will govern how this information is stored and disseminated. In general, this information must be protected from access by any unauthorized individual and may be communicated from the clinical laboratory that performed the test only to the ordering physician or his/her designee in accordance with state and federal laws. In most cases, the ordering physician then shares this information with patients and medical staff who are directly involved in the case. For analyses of nonhuman species and research applications, a variety of federal and state laws and regulations, policies of funding agencies and institutional rules and regulations may impact how this information is stored and disseminated.

In some embodiments, transcriptome based CNV screening of human embryos may be performed as a clinical diagnostic test. After information about specific genetic alterations is reported to the ordering physician, a medical professional may take one or more actions that can impact the assisted reproductive treatment plan or the subsequent testing or interventions performed on the embryo or its subsequent developmental stages. Additionally, the findings may provide actionable genetic information to the patient or patients from whom the embryo was generated. For example, a medical professional can record information in the parents' medical record regarding the embryo's risk of having a CNV that may be associated with prenatal loss or postnatal disability and/or mortality. In some embodiments, this information may prevent the use of this embryo to establish a pregnancy. In other circumstances, this information may provide evidence for risks for disease or disability at later stages of development that warrant subsequent medical tests and interventions should the embryo be transferred and lead to establishment of a pregnancy. In some embodiments, a medical professional may provide a copy of these test results to other medical specialists.

In other embodiments, this testing may be performed for nonclinical purposes. In some embodiments, this testing may be used for research applications on human embryos to advance research pertaining to the understanding of embryo genetics and biology and improving methods to generate and evaluate embryos. In other embodiments, these analyses may be used for diagnostic purposes on nonhuman embryos. In some cases, this testing will be used for similar purposes of screening for CNVs in preimplantation embryos of other mammals, including many domestic species. In other cases, this testing may be used in to advance biomedical research. In these applications, the scientists and staff directly involved in the experiments will have access to the information. For human embryo research, the data will be deidentified. In some embodiments, the significant results from these analyses may be presented to other scientists or the lay community in the form of publications and/or presentations.

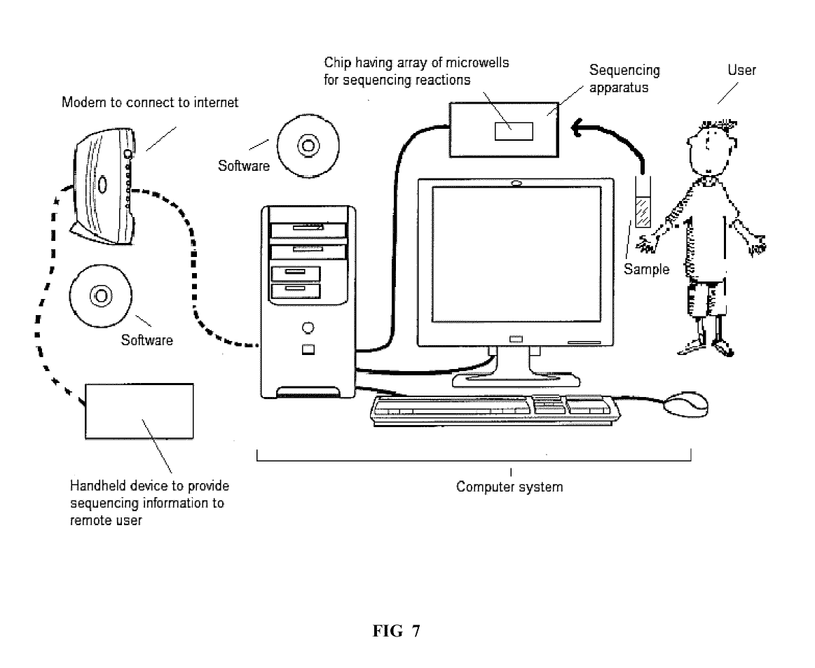

Any appropriate method can be used to communicate information pertaining to this analysis to another person. For example, information can be given directly or indirectly to a professional, and a laboratory staff member can input the report of embryo's genetic alteration into a computer-based record. In some embodiments, information is communicated by making a physical alteration to medical or research records. For example, a medical professional may make a permanent notation or flag a medical record for communicating the risk assessment to other medical professionals reviewing the record. In addition, any type of appropriate communication can be used to communicate the risk assessment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional. An exemplary diagram of computer based communication is shown in FIG. 7.

### XIII. EXAMPLES

It will be understood by those of skill in the art that numerous and various modifications can be made to yield essentially similar results without departing from the spirit of the present disclosure. All of the references referred to herein are incorporated by reference in their entirety for the subject matter discussed. The following examples are included for illustrative purposes only and are not intended to limit the scope of the disclosure.

### Example 1

### Demonstration of a High Correlation Between Copy Number and Locus Expression in Preimplantation Embryos

In this example, the effects of aneuploidy on the transcriptome of preimplantation mouse embryos were evaluated. Despite the incredibly high prevalence of aneuploidies and large genomic imbalances that are observed in human preimplantation embryos, little is understood about the biologic effects of these abnormalities. One of the central unanswered questions pertaining to these large genomic imbalances has been how copy number alterations impact the expression of the involved loci. In a variety of cancer cells and cells obtained from a variety of aneuploidies at later prenatal and postnatal periods, it has been shown that there is a general correlation between copy number and locus expression level. That is, gains typically cause increases in the expression of involved loci, and losses cause decreases in expression. It has been unclear whether this correlation also pertains to the early embryo for several reasons. First, studies of ploidy in preimplantation mouse embryos have found a lack of correlation between haploid copy number and locus expression levels. A study of haploid mouse embryos found roughly the same level of transcripts as diploid embryos (Latham, et al. (2002) Biology of Reproduction 67: 386-392, incorporated herein by reference). Tetraploid mouse embryos were also found to have similar expression levels of loci when compared to diploid embryos (Kawaguchi et al Kawaguchi, et al. (2009) Journal of Reproduction and Development 55: 670-675, incorporated herein by reference). Second, it is well established that mammalian embryos, in contrast to most other developmental stages or cell types, can develop apparently normally through the entire preimplantation period with many different large genomic imbalances such as aneuploidies. A striking example of the embryo's unique tolerance for genomic imbalances was recently demonstrated in a study that revealed that many of the genomic imbalances identified in human preimplantation embryos were not able to be perpetuated in embryonic stem cells derived from these embryos (Biancotti, et al. (2012) Stem Cell Research 9: 218-224, incorporated herein by reference). One possible explanation was that these genomic imbalances have little or no biologic effect on embryos, presumably due to little impact on the transcriptome. These studies serve as the first rigorous evaluation of the impact of aneuploidies on the transcriptome of the preimplantation embryo.

### Methods

Generation of Animals.

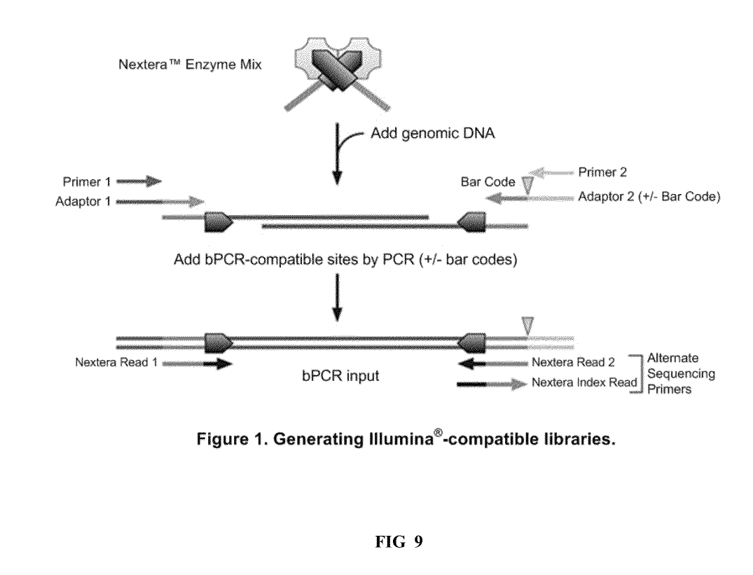

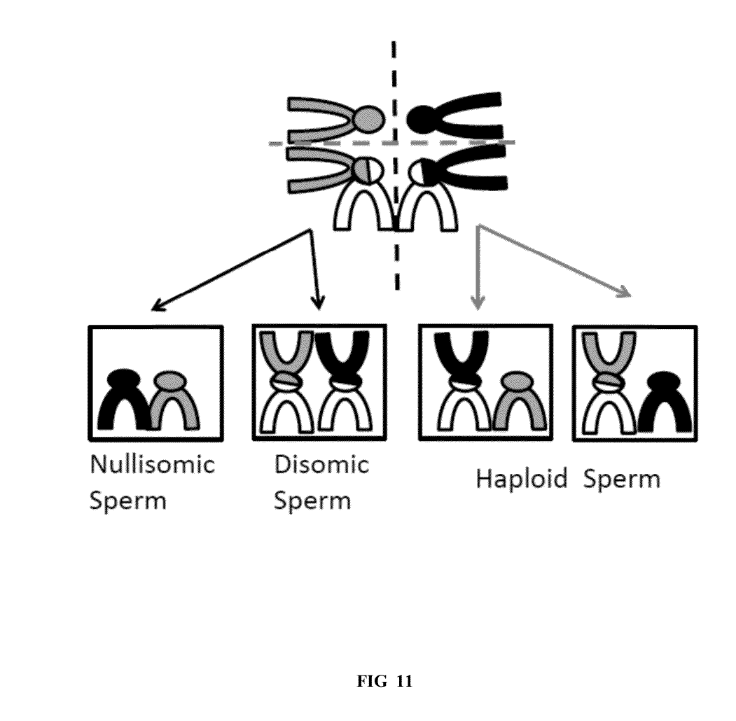

Large numbers of mouse embryos with whole chromosomal aneuploidies can be produced by using a sire that carries two Robertsonian (Rb) chromosomes, chromosomes formed by centromeric fusion of 2 acrocentric or telocentric, that share a common chromosomal arm, known as monobrachial homology (FIG. 9). During meiosis, segregation between these two Rb chromosomes is impaired, leading to the production of gametes and embryos that are aneuploid (monosomic or trisomic) for the chromosome on the common arm as shown in FIG. 11. For this study, male mice doubly heterozygous for 3 pairs of Rb chromosomes with monobrachial homology for chromosomes 10,11 and 15 were used to generate embryos. Fluorescent in situ hybridization of sperm from these males showed aneuploidy rates for the common arm chromosome of 40% with roughly half being nullisomic and half being disomic.

Embryo Production, Culture and Biopsy.

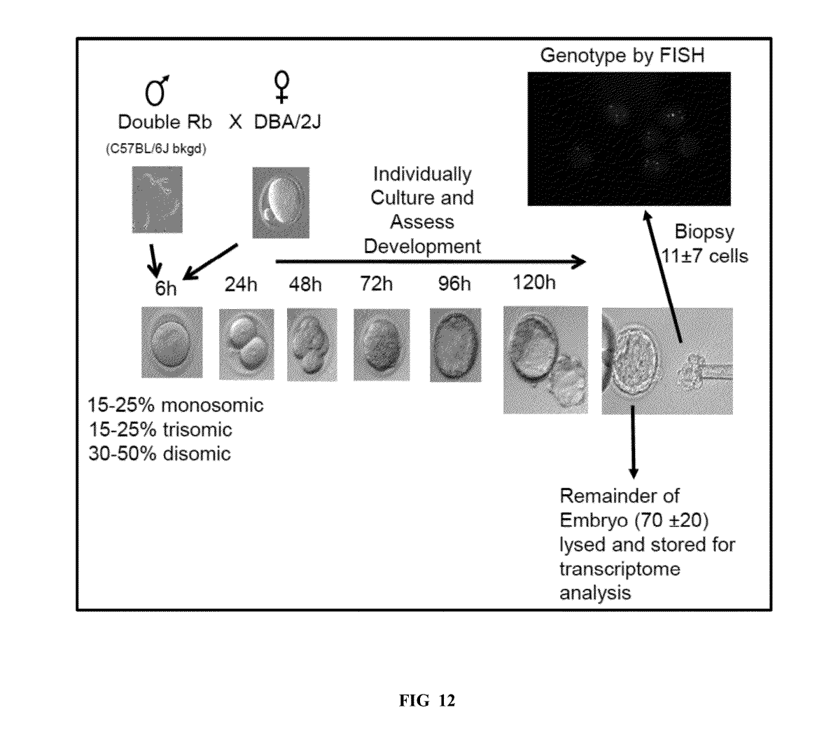

Embryos were generated by in vitro fertilization using cryopreserved sperm from males that carried the double Rb chromosomes in a C57B1/6J inbred background and oocytes from the DBA/2J inbred background (FIG. 12). Embryos were cultured individually in microdrops of a modified G series version 2 medium (Johnson, et al. (2009) RBM Online 19: 79-88, incorporated herein by reference) with daily morphologic assessment and culture medium changes. At 120 hours post-fertilization, 11+/−7 cells were removed from the mural trophectoderm of blastocysts using micromanipulator-controlled pipets and a Zylos-tk laser attached to an inverted microscope. The biopsy sample was processed for fluorescent in situ hybridization (FISH) using the protocol of Dozortsev and McGinnis (2001) Fertil Steril 76: 186-8 incorporated herein by reference. The remainder of the blastocyst was placed into Arcturus Picopure Extraction buffer, flash frozen in liquid nitrogen and then stored at −80 C until further processing.

Embryo Genotyping.

Biopsy samples fixed to slides were evaluated by FISH using BAC probes that anneal to the monobrachial chromosome as well as one other chromosome involved in the translocation using methods described by Scriven and Ogilvie (2010) Methods in Molecular Biology: Fluorescence in situ Hybridization (FISH) 659: 269-282. These probes were labeled with different fluorophores, and the biopsy samples were scored for signals from the two probes (first—from the Rb common arm chromosome and second from a chromosome on another Rb arm): 2/2-euploid, 3/2-trisomic, 1/2-monosomic, 3/3-triploid and mosaic when cells were present with different numbers of signals.

RNA-Seq Sample Preparation and Sequencing.

To evaluate the effects of the 3 trisomies on the transcriptome, 4-6 embryos of the same genotypes (disomic and trisomic) were pooled to serve as sources of RNA for this study (monosomic embryos were not evaluated because of insufficient numbers of embryos). Triplicate pools of disomic and trisomic embryos that were matched in terms of having the same number of embryos from the same IVF/culture run, the same parents, and similar developmental staging were generated for each of the 3 different trisomies. RNA was isolated using the Arcturus picopure kit per manufacturer's protocol, yielding 1-2 nanograms of high quality total RNA (RNA integrity number >8). Half of the RNA was amplified using the primer isothermal amplification method (Nugen Ovation RNA-Seq kit) to generate amplified cDNAs. This system produced over 4 micrograms of double-stranded cDNA from each sample. The cDNAs were fragmented with the Covaris Adaptive Focused Acoustics system and libraries were prepared using the Nugen Encore NGS Library Multiplex System 1. Libraries were generated with 4 different indexing tags to allow 4 libraries to be run per flow cell. Libraries were single-end sequenced on the Illumina HiSeq 2000.

Sequence Analysis.

Sequence quality was assessed with FastQC version 0.10.0 (http://www.bioinformatics.babraham.ac.uk/projects/fastqc/). Reads were aligned to the mouse genome (mm9) with TopHat version 1.3.1 (Trapnell, et al. (2009) Bioinformatics 25: 1105-1111, incorporated herein by reference) using the default parameter settings. Differential expression was assessed using the Cuffdiff utility in Cufflinks (Trapnell, et al. (2012) Nat Protoc 7: 562-578; Trapnell, et al. (2010) Nat Biotechnol 28: 511-5, incorporated herein by reference) in conjunction with a locally developed perl script. Density, box, and scatter plots to confirm comparability of datasets were generated using the Cummerbund program in the Cufflinks package.

### Results

Impact of Aneuploidies on Embryonic Development.

Genotyping of blastocysts revealed that 15-22% were trisomic (comparable to sperm disomy rates of 22-25%). For the monosomies, there were significantly reduced number of monosomic embryos for chromosomes 10 and 11 as compared to the frequencies of trisomies, whereas there was no difference for chromosome 15 (12 vs 15%). A small fraction, 4-7%, of embryos were noted to be mosaic, with most being a mix of the aneuploid and euploid cells. In reviewing the developmental progression and morphology of embryos, it was also found that there was no appreciable difference in development or morphology between embryos with any of the 3 trisomies or monosomy 15 and wild type (euploid) embryos.

RNA-Seq Analysis.

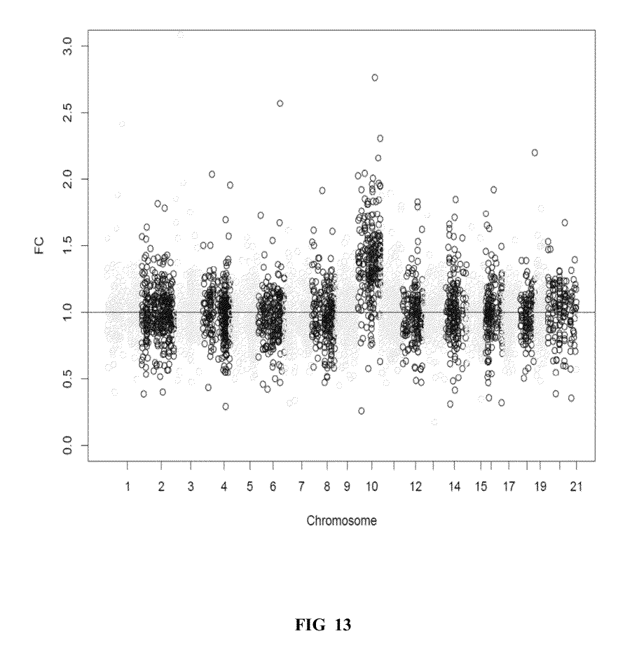

High throughput sequencing yielded on average 29.7 million 55-nucleotide reads per sample (min: 21.6 m, max: 38.6 m). QC analysis found all parameters assessed were good, with the exception of aberrant GC content and excess kmer content over approximately 10 bases at the 5′ ends of the reads. Based on this result, the first 10 bases from each read was trimmed using a locally developed perl script, yielding very high quality, 45-nucleotide reads for input to the aligner. Differential expression analysis using criteria of a fold change of greater than 1.5 and an FDR<0.05 found no differentially expressed transcripts for all 3 of the trisomies relative to the counterpart euploid samples. When the levels of expression of the transcripts on the trisomic chromosomes were compared to expression levels of the same loci in disomic samples, it was found that a significantly high fraction, exceeding 90% of transcripts, were overexpressed relative to disomic samples (χ-square<0.001). In contrast, there was no difference in levels of expression for nontriplicated loci between trisomic and disomic samples. The median/mean fold-change in expression for loci on the trisomic chromosome relative to expression levels of these loci in disomic samples was around 1.4 for all 3 trisomies. A graphical presentation of these fold changes for trisomy 10 is shown in FIG. 13.

Discussion: Genotypic analyses of embryos reveal that there was no selection against sperm or embryos with the 3 trisomies and monosomy 15 throughout the preimplantation period whereas the other 2 monosomies were compromised in their ability to develop throughout the preimplantation period. These findings support the clinical observation that trisomies often do not compromise preimplantation development whereas monosomies do (Sandalinas, et al. (2001) Hum Reprod 16: 1954-8, incorporated herein by reference). These findings also highlight the fact that, like with human embryos, embryos with substantial genomic abnormalities that are not compatible with prenatal development can develop essentially normally throughout the preimplantation period. These finding suggest that morphologic and developmental assessments have poor predictive value in identifying embryos with at least some genomic imbalances, including select trisomies.

The findings of no differentially expressed genes between trisomic and disomic RNA-Seq samples reveals that the standard means of assessing differential expression are too stringent for identifying primary or secondary perturbations in the transcriptome caused by aneuploidies. This fits with the general observation that aneuploidies cause relatively small magnitude changes that would require very large datasets to identify.

The high proportion of transcripts from the trisomic chromosome that are upregulated by approximately 1.5-fold indicates that there is a very strong correlation between copy number and transcript expression level in the preimplantation period, perhaps even higher than in most other cell types. To our knowledge, these are the first data that reveal this correlation. This finding is the basis for the approach of identifying aneuploidies in early embryos through analysis of the transcriptome.

### Example 2

### Detection of Aneuploidy in Embryos by Transcriptome Profiling

In this prophetic example, established approaches for generating RNA-Seq data from single cells and algorithms for identifying CNVs are applied in a likely clinical scenario. In this example, a father age 47 and a mother age 42 who have a 2 year history of 3 miscarriages are undergoing IVF and transcriptome-based CNV screening to reduce the chances of having an aneuploid pregnancy. Prior workup for recurrent miscarriages, including karyotypic analysis of both parents, is normal.

### Methods

Embryo Generation and Sample Acquisition.

Embryos are generated by standard ART procedures performed in a CLIA-certified ART laboratory, including controlled ovarian hyperstimulation, oocyte retrieval by follicular aspiration, fertilization by ICSI and culture of embryos to the blastocyst stage. On the 3^{rd} day of culture, the zona pellucida is breached in each developing embryo. On the 5^{th} day of culture, hatching and fully expanded blastocysts are transferred to individual, labeled microdrops on low profile biopsy dishes containing microdrops of G-MOPs overlaid with Ovoil. A herniated piece of trophectoderm from a hatching blastocyst or a piece of mural trophectoderm from an expanded blastocysts containing 5-10 cells is obtained using a Xylos tk laser and polar body biopsy pipets (Humagen) Immediately following biopsy, the blastocyst is transferred back to culture medium and returned to an incubator to continue the culture. Following completion of biopsies, all biopsied embryos are cryopreserved using a standard vitrification technique.

RNA Isolation and Spike in Control Addition.

Before lysis, each biopsy is washed three times through phosphate-buffered saline containing 5 mg/ml molecular biology grade bovine serum albumin using pipets that have an inner diameter that are close to the size of the biopsy sample (generally in the 1-5 micron range). Each washed biopsy sample is then placed in 3 microliters of hypotonic lysis buffer consisting of 0.2% Triton X-100 and 2 U/microliter of ribonuclease (RNase) inhibitors (Clontech, 2313B) in RNase free water in 0.2 microliter non-stick, RNAse-free, tubes (Ambion). This reaction buffer is included in the Clontech SMARTer™ Ultra Low RNA Kit. To each sample, 1 microliter of lysis buffer containing 10,000 copies of ERCC spike in synthetic RNA is added. Samples are then either snap frozen in liquid nitrogen or immediately processed for transcriptome analysis. Snap frozen samples are stored at −80 C or colder temperatures until subsequent processing.

Production of Double-Stranded cDNA.

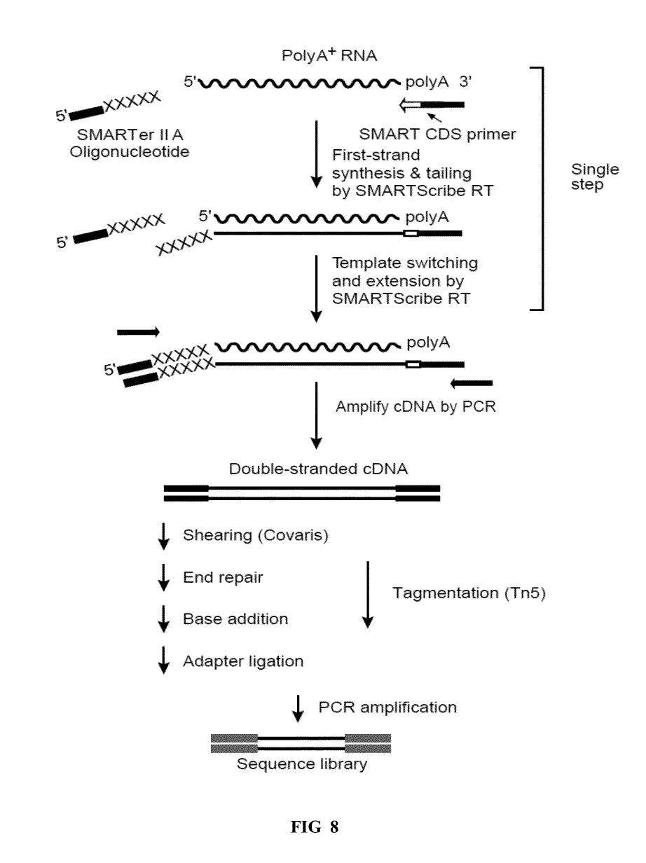

Samples are prepared and analyzed in a CLIA certified, CAP accredited laboratory. Both the first and second strands of cDNA are synthesized simultaneously using the template strand switching approach (Zhu, et al. (2001) Biotechniques 30: 892-897, incorporated herein by reference) by adding a reaction mix directly to the sample lysate. For this process, an oligodT primer is used by Moloney murine leukemia virus (MMLV) reverse transcriptase to reverse transcribe the first strand. Following completion of the reverse transcription, a polycytosine tract is added to the strand due to MMLV's terminal transferase activity. By also including a primer that has a sequence that is complementary to this polyC tract, the RT will then use this primer to extend the second strand (FIG. 8). This process is referred to as SMART (switch mechanism at the 5′ end of RNA templates). Poly(A)⁺ RNA is reverse-transcribed through tailed oligo(dT) priming using a cDNA synthesis (CDS) primer (5′-AAGCAGTGGTATCAACGCAGAGTACT(30)VN-3′, where V represents A, C or G) directly in total RNA or a whole cell lysate using Moloney murine leukemia virus reverse transcriptase (MMLV RT). First-strand cDNA generation is carried out with the addition of 5× First Strand Buffer (250 mM Tris-HCl pH 8.3, 375 mM KCl and 30 mM MgCl₂), dithiothreitol (100 mM), dNTP mix (10 mM), RNAse inhibitor, oligos (CDS primer and SMARTer II A oligo) and SmartScribe Reverse Transcriptase in a total volume of 10 microliters (see Clontech manual for details). Once the reverse transcription reaction reaches the 5′ end of an RNA molecule, the terminal transferase activity of MMLV adds a few nontemplated C nucleotides to the 3′ end of the cDNA. The carefully designed SMARTer II A oligo (5′-AAGCAGTGGTATCAACGCAGAGTACATrGrGrG-3′, where r indicates ribonucleotide bases) then base-pairs with these additional C nucleotides, creating an extended template. The reverse transcriptase then switches templates and continues transcribing to the end of the oligonucleotide. The resulting full-length cDNA contains the complete 5′ end of the mRNA as well as an anchor sequence that serves as a universal priming site for second-strand synthesis. Following cDNA synthesis, the products are purified using SPRI Ampure Beads. The reagents for this method are available in the Clontech SMARTer™ Ultra Low RNA Kit.

cDNA Amplification.

Double stranded cDNA produced by the SMART technology contains sequences at each end of the cDNA that serve as a universal priming sites for amplification by PCR. PCR-based amplification is performed using the long-distance PCR kit, Advantage 2 (Clontech) with PCR primer (5′-AAGCAGTGGTATCAACGCAGAGT-3′) and thermocycling conditions: 15 cycles of 95° C. for 15 seconds, 65° C. for 30 seconds and 68° C. for 6 minutes. This protocol should produce 2-7 nanograms of DNA with the predominant species ranging in size from 400-9000 bp with a peak at approximately 2000 bp. The amplification products should evaluated using a nanodrop spectrophotometer and the Agilent 2100 BioAnalyzer using the nanochip.

DNA Fragmentation.

DNA is fragmented using the Nextera technology, which utilizes a tn5 transposase to simultaneously fragment the double-stranded DNA and ligate adapters to the ends of the fragments (FIG. 9). With the Tn5 protocol, the amplified cDNA is ‘tagmentated’ at 55° C. for 5 min in a 20 μl reaction with 0.25 μl of transposase and 4 μl of 5×HMW Nextera reaction buffer (containing Illumina-compatible adapters). To strip the transposase off the DNA, 35 μl of PB is then added the tagmentation reaction mix, and the tagmentated DNA was purified with 88 μl of SPRI XP beads (sample to beads ratio of 1:1.6). The reagents for this method are available in Nextera DNA sample kits (Epicentre/Illumina).

Library Production.

Libraries are prepared for sequencing using the Illumina platform. Limited-cycle PCR with a four-primer reaction adds bridge PCR (bPCR)-compatible adaptors to the core library (used for binding fragments to the flow cell). By including different Illumina compatible bar codes between the downstream bPCR adaptor and the core sequencing library adaptor in sets of 4 samples, it is possible to run 12 samples on the same flow cell. The bPCR/barcode/sequencing adapters are added to the library by incubating the reactions at 72° C. for 3 minutes followed by 9 cycles of: 95° C. for 10 seconds; 62° C. for 30 seconds and 72° C. for 3 minutes. The reagents for this step are included in the Nextera DNA Sample Prep Kit (Illumina-compatible). Following amplification, library quality is confirmed using DNA 1000 kits on an Agilent Bioanalyzer.

Sequencing.

Twelve samples are run per flow cell on the Illumina HiS eq 2000 system to generate single end reads of 55 bp. These parameters are expected to generate about 10 million reads/sample. In a report using this method for single cell RNA-Seq, it was found that at above 3 million uniquely mapping reads, there was little impact on transcript detection (Ramskold, et al. (2012) Nat Biotechnol 30: 777-82, incorporated herein by reference).

Quality Assessment and Data Filtering.

FastQC version 0.10.0 (http://www.bioinformatics.babraham.ac.uk/projects/fastqc/) is used to assess quality per sequence and per base (phred scores); GC and N content; sequence length distribution, overrepresented sequences, sequence duplication levels and kmer content. Based on these quality scores, poor sequences and/or segments of sequence are culled.

Sequence Alignment and Depth of Coverage Assessment.

Novoalign from Novocraft Short Read Alignment Package (http://www.novocraft.com/index.html) is used to align each lane's SEQ file to the reference genome. Human Genome reference sequence (GRCh37.p11, release Dec. 23, 2012), is indexed using novoindex program (−k 14 −s 3). The output format was set to SAM and default settings were used for all options. Using SAMtools (http://samtools.sourceforge.net/), the SAMfiles of each lane were converted to BAM files, sorted and merged for each sample and potential PCR duplicates were removed using Picard (http://picard.sourceforge.net/) (Li et al., 2009). To retrieve the depth of coverage information of each base, a PILEUP file for each sample is generated using SAMtools and the average coverage per capture interval is calculated using a custom script

SNP Genotyping.

Before identifying heterozygous SNPs in the genome, the depth of coverage for each base, an important parameter in determining the confidence for calls is calculated from a PILEUP file generated by SAMTools software. Variant sites are then called by the Genome Analysis Toolkit software (McKenna, et al. (2010) Genome Res 20: 1297-1303, incorporated herein by reference).

CNV Identification Using Locus Expression Data.

CNVs will be identified using ExomeCNV (FIG. 11; Sathirapongsasuti, et al. (2011) Bioinformatics 27: 2648-2654, incorporated herein by reference). This program uses a normalized depth of coverage ratio to evaluate the relative expression at the exon level of the sample as compared to the reference. The reference is the median read counts for each exon obtained from a large dataset of embryonic samples that are generated in the same manner as the test sample. Using ExomeCNV, a CNV in an exon is identified by a deviation of the transformed ratio from the null, standard normal distribution that is beyond empirically defined thresholds. Once exons are evaluated, the exonic data are combined into segments using circular binary segmentation (CBS).

CNV Identification Using Allelic Expression Data.

For this example, it is assumed that the haplotypes of the embryo have not been determined ExomeCNV is also used for identifying significant skewing in the allelic ratios. This program first evaluates the allele frequencies of heterozygous SNPs in the sample to determine if there is a deviation from expected frequencies. For this analysis, the frequency of the non-reference, ‘B’ allele is determined for each polymorphic SNP: B allele frequency (BAF) at position i is calculated by # Bi (# reads of B allele) divided by Ni (the total number of reads or depth-of-coverage). The expected frequencies for the B allele frequencies for autosomes and the X chromosome in females are 0.5 and 0 or 1 for the X and Y chromosomes in male embryos. To evaluate differences for polymorphic position, it is determined whether the binomial rejects the null hypothesis (i.e., no difference from the expected frequencies). Segmentation of individual SNP data are then done using a circular binary segmentation algorithm by determining whether there is a significant increase in variance of BAFsample from that of BAFreference using an F-test for equality of variance. This reference is composed of median BAFs values for the same dataset as described for locus based CNV detection.

### Expected Results

The results from transcriptome profiling of samples from 6 embryos show that 4 embryos have no regions identified as being abnormal based on comparisons of locus and allele expression data to the reference, one embryo has increased expression of and altered AERs for loci on chromosome 21, indicating trisomy 21 and one embryo has increased expression of and altered AERs for loci on chromosome 16, indicating trisomy 16. The results from the clinical laboratory are conveyed to the ordering physician and after consultation with the family, it is decided that only one of the embryos without evidence of CNVs should be warmed and then transferred during a natural cycle. The remaining 3 embryos without evidence of CNVs are to be stored for subsequent transfers if the couple so desires. The two cryopreserved embryos with evidence of CNVs are donated to research.

### Example 3

### Detection of a Segmental Aneusomy Using Transcriptome Profiling

In this example, embryos are screened for causative deletion in a parent who has velocardiofacial syndrome (VCFS). VCFS is an autosomal dominant contiguous gene syndrome that is most commonly associated with congenital heart disease, palatal abnormalities, learning difficulties, immune deficiency and characteristic facial features. This disorder affects 1 in 4000-6000 births. More than 85% of patients, including the father in this example, have a 2.5 megabase deletion in region 22q11.2. The parents opt for preimplantation genetic diagnosis to reduce their chances of having a pregnancy carrying this deletion. Upon considering diagnostic approaches, they opt for transcriptome-based screening as they also wish to have generalized aneuploidy screening.

### Methods

The methods for embryo, sample and data generation will be the same as described above in Example 2. The CNV detection approach described in Example 2 will identify aneuploidies. To determine whether embryos carry the VCFS deletion, the results from CNV screening will be examined to determine if embryos carry the deletion from 17-20 Mb on proximal 22q.

### Expected Results

In this example, 7 blastocysts are biopsied. CNV screening reveals that 4 embryos are unlikely to carry the 22q11.2 deletion and 3 are likely to have the deletion, due to both a decrease in expression of loci in this region and LOH based on evaluation of allelic expression. CNV screening of the embryos without evidence of the 22q11 deletion also reveals one has evidence of trisomy 22, one has trisomy 5 and one has trisomy 8 and trisomy 15. Based on these results, a decision is made by the healthcare team and parents to transfer one of the cryopreserved blastocysts that does not have evidence of the 22q11 deletion or other CNVs. The remaining embryos are maintained in cryopreservation until decisions are made about their respective fates.

### Example 4

### Detection of Uniparental Disomy by Transcriptome Profiling

In this example, a female carrier of a 13;14 Robertsonian translocation and her husband are referred for preimplantation genetic diagnosis after over 4 years of trying to have a child. Carriers of this translocation are at high risk of producing aneuploid conceptuses. The couple chooses to undergo transcriptome profiling-based CNV detection embryo screening to increase their chances of establishing a chromosomally normal pregnancy.

### Methods

The methods for this application are described in Example 2.

### Expected Results

In this example, 9 embryos are biopsied and cryopreserved. CNV screening results provide evidence for 2 embryos having trisomy 13, one having monosomy 13, two having trisomy 14, one having monosomy 14, two having no evidence of CNVs and one with uniparental disomy (UPD) 14. UPD 14 is detected by there being no evidence of abnormalities in the expression of loci on chromosome 14, but evidence for LOH for loci on chromosome 14 based on allelic expression. The mechanism of UPD in this case is likely to arise as a result of the formation of a zygote with trisomy 14 followed by ‘trisomy rescue’ in which the paternal chromosome 14 is lost. Based on these results, a decision is made by the healthcare team and parents to transfer an embryo with no evidence of CNVs.

### Example 5

### Screening for a Single Gene Disorder in Concert with CNV Screening

In this example, a male with congenital bilateral absence of the vas deferens and his wife are planning to undergo preimplantation genetic screening for mutations in the cystic fibrosis gene (CFTR). Absence of the vas deferens causes male infertility and is most commonly caused by mutations in the CFTR gene. Mutations in the CFTR also cause cystic fibrosis (CF), an autosomal recessive disease associated with a variety of disorders, including pulmonary and pancreatic dysfunction. Approximately 1 in 25 Caucasians carry a mutation in CFTR. Workup for CBAVD reveals that the male is a compound heterozygote, carrying ΔF508, the most common mutation in the CFTR gene, and another mutation R117H. Testing of the wife reveals that she also carries the ΔF508 mutation. Homozygosity for ΔF508 leads to classic cystic fibrosis. This couple opts to have PGD as part of their assisted reproduction to reduce the chances of having a pregnancy affected by CF. The couple chooses transcriptome-based method as they also wish to reduce their chances of having a pregnancy with a large genomic imbalance. The CFTR gene is known to be expressed in the blastocyst and plays an important role in formation of the blastocoel.

### Methods

CNV screening is performed as described in Example 2. For mutation screening, the coding sequences of the CFTR transcripts are examined in detail, looking for presence of the 2 mutations found in the parents: c.1521_{—}1523 delCTT, a 3 basepair mutation in exon 11 that causes the ΔF508 mutation and c.305G>A in exon 4, a single basepair transition that causes the R117H mutation in the CFTR protein. The CFTR transcribed sequences are scanned for other alterations in the CFTR transcript as well. The CFTR transcript sequences are evaluated for sequence variants and calls are made using the genome analysis toolkit.

### Expected Results

Five blastocysts are biopsied and cryopreserved. CFTR mutation analysis reveals one embryo to be homozygous for the ΔF508 mutation, two embryos to be compound heterozygotes for the ΔF508 and R117H mutations and two embryos to be carriers of the R117H mutation. CNV analysis reveals that one of the ΔF508 carrier embryos also contains trisomy 16 and one R117H carries a trisomy 20. Based on these results, a decision is made by the healthcare team and parents to transfer the embryo that carries the R117H mutation and has no evidence of CNVs.

### Example 6

### CNV Screening and Linkage Analysis

In this example, an African-American couple who are both carriers of the sickle cell mutation (HbSS mutation) decide to use ART & PGD to prevent having a pregnancy affected with sickle cell disease, an autosomal recessive disorder that is characterized by intermittent vaso-occlusive events and chronic hemolytic anemia. They have one affected child. In considering options, the couple choose to use transcriptome-based linkage analysis and CNV screening to reduce the risks of establishing a pregnancy affected by sickle cell disease or aneuploidy.

### Methods

The haplotypes of the parents and the affected child are first determined by genotyping these individuals. Genomic DNA is isolated from peripheral blood samples using the QIAmp DNA mini blood kit (Qiagen). The individuals are genotyped using an Illumina custom SNP microarray that has been developed to genotype all SNPs in coding regions of all transcripts expressed in human embryos. The haplotypes for the three individuals are generated using Triocaller software (Chen, et al. (2013) Genome Research 23: 142-151, incorporated herein by reference). Embryos are screened for CNVs as described in Example 2. SNP genotype data are generated using the genome analysis toolkit. Multipoint linkage analysis for the parents and embryos is performed using SNPLINK software (Webb, et al. (2005) Bioinformatics 21: 3060-3061, incorporated by reference herein)

### Expected Results

Haplotype analysis identified multiple informative SNPs that are closely linked to the HbSS alleles in both parents. Six embryos are biopsied and cryopreserved. Linkage analysis reveals that two are found to be HbSS homozygotes, 3 are HbSS heterozygotes and 1 is homozygous unaffected. CNV analysis reveals that one of the HbSS heterozygotes has evidence for trisomy 7 and the unaffected embryo has evidence for trisomy 18. The results are conveyed to the healthcare provider. Based on these results, a decision is made by the healthcare team and parents to transfer a HbSS carrier embryo without evidence of large CNVs.

### Example 7

### CNV Screening and Screening for an Imprinting Disorder

A couple who are undergoing IVF for fertility treatment are very knowledgeable about the potential adverse outcomes from IVF. They express their wish to screen embryos for large CNVs and for abnormalities in genomic imprinting that are associated with Beckwith Wiedemann syndrome (BWS). BWS is a growth disorder characterized by a number of malformations and an increased risk for embryonal tumors. This disorder arises from an increased expression of genes in 11p15.5 that are normally expressed from the paternal chromosome. Children of subfertile parents conceived by assisted reproductive technology appear to have about a 9-fold increased risk for this disorder.

### Methods

CNV screening methods are performed as described in Example 1. For evaluating imprinting of the BWS region, the expression of the parental alleles of 13 loci in the 11p15.5 region including KCNQ1OT1 and CDKN1C are evaluated using allele-specific SNPs. In the normal situation, the paternal haplotype should express KCNQ1OT1 and not any of the neighboring loci whereas the KCNQ1OT1 should not be expressed and all of the neighboring alleles should in the maternal allele. The identification of skewing of AERs in this region consistent with these normal patterns of gene expression would indicate that this chromosomal region is normally imprinted. In cases in which there is overexpression of the genes that are normally expressed from this region following paternally inheritance, there is an increased risk for BWS.

### Expected Results

Eight embryos are biopsied and cryopreserved. All are found to have the normal pattern of allelic expression in the 11p15.5 region associated with BWS, suggesting that the likelihood of BWS developing from these embryos would be very low. CNV screening identifies 3 embryos without evidence for CNVs. Based on these results, a decision is made by the healthcare team and parents to transfer one of these embryos to establish a pregnancy.

### Example 8

### CNV and Genetic Fingerprinting

In this example, a cohort of embryos from a couple is being evaluated by morphologic analyses using time-lapsed imaging as part of an IRB-approved study. The couple indicates that they also wish to have transcriptome-based CNV screening. The IVF cycle yields 4 blastocysts, of which 2 are found to have no evidence of CNVs based on transcriptome-based CNV screening as described in Example 2. Due to the maternal age of 42 and the parents' strong wishes, a decision is made to transfer 2 embryos. At midgestation, only one fetus is present. To track the outcomes of the two transferred embryos, it is decided that the unique genetic identities of the embryos would be used to determine which embryo produced the fetus.

### Methods

A sample from the amniocentesis was sent for SNP genotyping using the same custom SNP array as described in Example 6. Fetal and embryonic genotypes are compared by calculating concordance rates of all SNPs (# SNPs with matching genotypes/total # SNPs). For the matching embryonic and fetal samples, the concordance should be >99% whereas those from a sibling would be in the range of 75%.

### Expected Results

Comparisons of SNP genotyping results from the fetus and embryos successfully identify which embryo is successfully developing as a fetus. These results allow for all embryonic data for these 2 embryos to be linked to their outcomes.

### Example 9

### Determination of Embryo Gender

In this example, a woman who is a carrier of a mutation in the DMD gene, the gene associated with Duchenne muscular dystrophy, wishes to use preimplantation genetic diagnostics to avoid having a boy affected by this X-linked disease. No other relatives are available for linkage analysis. The woman opts to proceed with transcriptome-based gender determination and CNV screening with the goal of establishing a pregnancy with a healthy female fetus.

### Methods

CNV screening methods as described for Example 2 are used. To determine the gender of the embryo, the expression profiles of the sex chromosomes are evaluated. First, it is determined if there is expression of Y-linked genes outside of the pseudoautosomal region. Second, the expression of X-linked genes outside of the pseudoautosomal region is evaluated. A gender of male will be assigned to embryos in which there is Y-linked gene expression and X-linked gene expression consistent with a single copy of this chromosome. A female gender will be assigned for embryos in which there is no evidence of Y-linked gene expression and expression levels of X-linked loci are consistent with 2 copies. Furthermore, SNP genotyping will reveal biallelic patterns for SNPs on the X chromosome.

### Expected Results

In this case, 7 blastocysts are biopsied and cryopreserved. Three embryos are found to have no evidence of CNVs. Of the 3 embryos without detectable CNVs, 1 is found to be male and 2 are female based on expression profiles from the sex chromosomes. Based on these results, a decision is made by the healthcare team and parents to transfer one female embryo without evidence of CNVs.

### Example 10

### CNV Screening and Developmental Potential

In this example, an infertile couple wishing to maximize the possibility for having a healthy child produced from the present IVF cycle opts to screen their embryos for CNVs and developmental potential using transcriptome data.

### Methods

CNV screening is performed as described in Example 2. For assessment of health and developmental potential, a dataset of transcriptome profiles from embryos that have no evidence of CNVs and are confirmed to produce healthy children will be developed. A composite profile, representing the median expression of loci from this dataset will be generated. This ‘developmentally competent’ reference profile will be used to prioritize and possibly even select embryos for transfer. To do this the transcriptome profile for the embryo will be compared to the developmentally competent reference using differential gene expression and pathway analyses. Embryos will be ranked according to their similarity to the developmentally competent reference profile. As the dataset grows and this algorithm is refined, it may be possible even to set thresholds that indicate a high probability of a poor outcome, thus defining a threshold for recommending against transfer. Embryos that are not found to have evidence for CNVs that contraindicate transfer using methods outlined in Example 2 will be further prioritized by comparisons to the developmentally competent profile.

### Expected Results

Six blastocysts are biopsied and cryopreserved. Four are found to have evidence of large CNVs. Comparisons of the transcriptome profiles for the two embryos without evidence for CNVs to the developmentally competent reference profile, identifies the embryo with the profile that more closely matches the developmentally competent reference. Based on these results, a decision is made by the healthcare team and parents to transfer the embryo with the transcriptome profile more closely related to the developmentally competent reference.

### Example 11

### CNV and Mitochondrial Mutation Analysis

In this example, a woman who has a mild form of the mitochondrial disease NARP (neurogenic muscle weakness, ataxia, retinitis pigmentosa) wishes to undergo preimplantation genetic analysis to have an unaffected or less severely affected child. Preimplantation diagnostics have shown that even though this mutation in the mitochondrial genome is maternally transmitted, the mutation load between embryos can vary considerably, with some even having no detectable mutation.

### Methods

CNV screening is performed as described in Example 2. To identify mitochondrial transcripts, reads will be mapped to the human mitochondrial genome using the same algorithms Sequence variants and read depths will be determined as described in Example 2. The NARP mutation arises from a guanine to thymine transversion at nucleotide position 8993. The read counts for the wild-type and mutant alleles will provide an indication of the degree of mutation in embryonic cells.

### Expected Results

Nine blastocysts are biopsied and 6 are found to have evidence of large CNVs based on CNV screening. Of the 3 embryos without detected CNVs, the percentages of mutant transcripts in the samples are estimated to be 5, 15 and 45%. Based on these results, a decision is made by the healthcare team and parents to transfer the embryo with no evidence of CNVs and the lowest mutation burden (5%).

### Example 12

### CNV Screening Combined with all Other Embryo Diagnostics

In this example, an infertile couple is interested in using any and all modalities for screening their embryos to provide the greatest possible chance of producing a healthy pregnancy from their IVF cycle. With that goal, the couple agrees to have transcriptome-based screening for CNVs, clinically significant mutations, genomic imprinting and developmental competence. In addition, noninvasive diagnostics of time-lapsed imaging of embryos and metabolomic and proteomic profiling of culture medium will be performed. This multifaceted assessment will provide a tremendous amount of information about the health and developmental potential of the embryos.

### Methods

The transcriptome analyses will be performed as described in Examples 2 (CNV screening), 5 (mutation screening), 7 (genomic imprinting) and 10 (developmental competence). Metabolic profiling will be performed through quantitative analysis of metabolites high performance liquid chromatography-mass spectrometry. Proteomic profiling will be performed using liquid chromatography-tandem mass spectrometry. These profiles are assessed by comparison to embryos that have successfully developed into liveborns much in the same way that developmental competence is assessed by transcriptome profiling. Time lapse imaging will be performed using the Eeva time-lapse imaging system (Auxogyn). This system analyzes cell division timing data for parameters that have been correlated with successful preimplantation development. For each of these analyses a developmental competence score is assigned that reflects the likelihood of a good outcome. An overall developmental competence score is then obtained by summing the scores for each test.

### Expected Results

Ten embryos are biopsied and cryopreserved. Of these, 6 have evidence for CNVs. Of the 4 that do not have detectable CNVs, these embryos are then ranked based on their overall developmental competence scores. Based on these results, a decision is made by the healthcare team and parents to transfer the embryo without evidence of CNVs and the highest overall developmental competence score.

1-150. (canceled)

151. A method for determining a presence or absence of a genomic copy number variation in a preimplantation embryo, the method comprising:
a. reverse transcribing RNA derived from a preimplantation embryo to form cDNA;
b. analyzing the cDNA to determine a presence or absence of the genomic copy number variation in the preimplantation embryo.

152. The method of claim 151, wherein the analyzing comprises performing high-throughput sequencing of the cDNA to generate sequence reads.

153. The method of claim 152, wherein the sequencing comprises whole transcriptome sequencing.

154. The method of claim 152, wherein the sequencing comprises partial transcriptome sequencing.

155. The method of claim 152, wherein the analyzing comprises enumerating the sequence reads.

156. The method of claim 152, wherein the analyzing comprises aligning the sequence reads to a reference genome.

157. The method of claim 152, wherein the analyzing comprises comparing a number of the sequence reads corresponding to one or more loci on a first chromosome to a number of the sequence reads corresponding to one or more loci on a second chromosome, wherein the first chromosome is suspected of exhibiting a copy number variation, and the second chromosome is euploid.

158. The method of claim 152, wherein the analyzing comprises normalizing a number of the sequence reads corresponding to one or more loci on a first chromosome suspected of exhibiting a copy number variation to generate a normalized chromosome count, and comparing the normalized chromosome count to a normalized chromosome count for a reference sample from one or more preimplantation embryos without a genomic imbalance.

159. The method of claim 152, wherein a number of the sequence reads corresponding to one or more loci on a first chromosome suspected of exhibiting a copy number variation is normalized to a number of the sequences reads corresponding to one or more loci on a second chromosome suspected of being euploid.

160. The method of claim 152, wherein a number of the sequences reads corresponding to one or more loci on a first chromosome suspected of exhibiting a copy number variation is normalized to a number of the sequence reads corresponding to loci on a plurality of chromosomes.

161. The method of claim 152, wherein the high-throughput sequencing comprises
a. bridge amplification and incorporation of four fluorescently-labeled, reversible terminator-bound dNTPs;
b. measurement of release of inorganic phosphate;
c. passing the cDNA through a nanopore; or
d. measuring hydrogen ion release during polymerization of cDNA.

162. (canceled)

163. The method of claim 151, wherein the analyzing comprises amplifying the cDNA.

164-170. (canceled)

171. The method of claim 163, wherein a plurality of preimplantation embryos is analyzed, and amplifying cDNA from the plurality of preimplantation embryos comprises indexing cDNA from each preimplantation embryo.

172-173. (canceled)

174. The method of claim 151, wherein the analyzing comprises comparing an amount of cDNA derived from one or more loci to an amount of cDNA derived from the one or more loci from one or more preimplantation embryos known to be euploid or disomic for the one or more loci.

175. The method of claim 151, wherein the analyzing comprises comparing an amount of cDNA derived from one or more loci to a median value of cDNA derived from the one or more loci from one or more preimplantation embryos known to be euploid or disomic for the one or more loci.

176. The method of claim 151, wherein the analyzing comprises comparing an amount of cDNA derived from one or more loci to a median expression value of cDNA derived from the one or more loci from a plurality of preimplantation embryos.

177. The method of claim 151, wherein the analyzing comprises comparing a normalized expression value for cDNA from one or more loci to an amount of cDNA derived from the one or more loci from one or more preimplantation embryos known to be euploid or disomic for the one or more loci.

178. The method of claim 151, wherein the analyzing comprises comparing a normalized expression value for cDNA from one or more loci to a median value of cDNA derived from the one or more loci from one or more preimplantation embryos known to be euploid or disomic for the one or more loci.

179. The method of claim 151, wherein the analyzing comprises comparing a normalized expression value for cDNA from one or more loci to a median expression value of cDNA derived from the one or more loci from a plurality of preimplantation embryos.

180. The method of claim 151, wherein the analyzing comprises determining a first ratio of an amount of cDNA derived from a first set of one or more loci to an amount of cDNA derived from a second set of one or more loci, and comparing the first ratio to a second ratio derived from one or more preimplantation embryos known to be euploid, wherein the second ratio is a ratio of an amount of cDNA derived from the first set of one or more loci to an amount of cDNA derived from the second set of one or more loci.

181-244. (canceled)


SEQUENCE LISTING (3 sequences)

SEQ ID NO: 1 - DNA, 57 bp, Artificial Sequence
  Description of Artificial Sequence Synthetic primer
aagcagtggtatcaacgcagagtacttttttttttttttttttttttttttttttvn

SEQ ID NO: 2 - DNA, 30 bp, Artificial Sequence
  Description of Artificial Sequence Synthetic oligonucleotide
aagcagtggtatcaacgcagagtacatggg

SEQ ID NO: 3 - DNA, 23 bp, Artificial Sequence
  Description of Artificial Sequence Synthetic primer
aagcagtggtatcaacgcagagt